(19) United States
(12) Patent Application Publication
(10) Pub. No.: US 2021/0246575 A1
(43) Pub. Date: Aug. 12, 2021

(54) METHODS AND DEVICES FOR MAKING NANOFIBERS AND NANOFIBER SCAFFOLDS

(71) Applicant: University of Georgia Research Foundation, Inc., Athens, GA (US)

(72) Inventors: Nataraja S. Yadavalli, Athens, GA (US); Darya Asheghali, Durham, NC (US); Sergiy Minko, Athens, GA (US); Alexander Tokarev, Mt Pleasant, SC (US)

(21) Appl. No.: 17/169,940

(22) Filed: Feb. 8, 2021

Related U.S. Application Data
(60) Provisional application No. 62/971,512, filed on Feb. 7, 2020.

(51) Int. Cl. D01D 5/06; C12M 1/12; D04H 3/04; D01F 6/62

(52) U.S. Cl. CPC D01D 5/06; D01F 6/625; D04H 3/04; C12M 25/14

(57) ABSTRACT

Provided are methods for forming single filament nanofibers, methods for forming 3D nanofiber scaffolds, apparatus for forming nanofibers and nanofiber scaffolds, and nanofiber cell culture scaffolds formed using the methods and devices. Single filament nanofibers (having a diameter of about 50 nm-100 μm) can be formed by gravitational drawing by dispensing a droplet of a polymer solution from a nozzle such that the droplet free falls from the nozzle onto a base, causing the polymer solution to be drawn into a fluid tail. Nanofiber scaffolds can be built by forming and collecting single filament nanofibers in an ordered manner on a collection frame to form 2D arrays that can then be stacked. The spacing and alignment of individual fibers is precisely controlled. Device for forming the 3D nanofiber scaffolds are provided. The 3D nanofiber scaffolds can be cell culture scaffolds having a porosity of 50% or greater.

### CROSS-REFERENCE TO RELATED APPLICATIONS

This application claims the benefit of and priority to U.S. Provisional Application Ser. No. 62/971,512, having the title “METHODS AND DEVICES FOR MAKING NANOFIBERS AND NANOFIBER SCAFFOLDS”, filed on Feb. 7, 2020, the disclosure of which is incorporated herein by reference in its entirety.

### BACKGROUND

Electrospinning process is a popular method to make nanofibers that utilizes high-voltage electrical field to stretch polymer droplets into a liquid jet, which generates nanofibers following solvent evaporation or cooling of the polymer melt. Instability is an inherent property of the jet, thereby allowing very poor control of fiber manipulation and 3D-alignment. There remains a need for methods and apparatus for making single filament nanofibers that can be manipulated and 3D-organized in complex structures with well-controlled porosity, spacing, angular orientation, dimensions and mechanical properties. These needs and other needs are satisfied by the present disclosure.

### SUMMARY

Embodiments of the present disclosure provide methods for forming single filament nanofibers, methods for forming 3D nanofiber scaffolds, apparatus for forming nanofibers and nanofiber scaffolds, nanofiber cell culture scaffolds, and the like.

An embodiment of the present disclosure includes a method of making single filament nanofibers. The method can include dispensing a droplet of a polymer solution from a nozzle onto a base. The nozzle is positioned above the base such that the droplet free falls from the nozzle onto the base, causing the polymer solution to be drawn into a fluid tail between the base and the nozzle. As the fluid tail is being drawn, a solvent of the polymer solution evaporates, thereby solidifying the fluid tail to form a nanofiber. The fiber has a fiber diameter of about 50 nm-100 μm and a fiber length from about 1 cm to 2 meters.

An embodiment of the present disclosure includes a method of forming a nanofiber scaffold by forming a first single filament nanofiber by gravitational drawing of a polymer solution from a nozzle to a base. The polymer solution can include a polymer and a solvent, where evaporation of the solvent during gravitational drawing causes the polymer solution to solidify into a fiber. Each nanofiber can independently have a diameter of about 50 nm-100 μm. The first single filament nanofiber can be collected on a collection frame. One or more subsequent single filament nanofibers can be formed then collected on a collection frame to form a first 2D array layer. Each single filament nanofiber can be aligned and spaced in the first 2D array layer, such that an alignment angle between any two adjacent single filament nanofibers can be independently selected from about 0 to 179 degrees, and a spacing between any two adjacent fibers can be independently selected from about 10 micron or larger.

An embodiment of the present disclosure includes a device for forming a 3D nanofiber scaffold. The device can include a fiber positioning assembly, a fiber drawing assembly, a fiber collection and delivery assembly, and a cutting tool. The fiber drawing assembly can have a nozzle positioned above a tension stage. The fiber collection and delivery assembly can include a moving stage and a fiber collector frame.

An embodiment of the present disclosure also includes a 3D cell culture scaffold formed from ordered 2D arrays of nanofibers and having a porosity of 50% or higher. Each of the nanofibers in the arrays can have a diameter between 50 nm and 2 μm. The nanofibers can be formed by gravitational drawing of a polymer solution from a nozzle to a base. The polymer solution can include a biocompatible polymer and a solvent, where evaporation of the solvent during gravitational drawing causes the polymer solution to solidify into a fiber. The nanofibers in the arrays can have controlled alignment and spacing. The arrays of nanofibers are layered to form the scaffold.

Other compositions, apparatus, methods, features, and advantages will be or become apparent to one with skill in the art upon examination of the following drawings and detailed description. It is intended that all such additional compositions, apparatus, methods, features and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1A is a diagram of the gravity-drawing concept. The droplet falls at speed v(t), drawing out a fiber. FIG. 1B is an example of a prototype nanofiber forming and scaffolding device (not to scale) in accordance with embodiments of the present disclosure.

FIGS. 2A-G are examples of GFD fabricated nano- and microfibers of diameter 460 nm (2A), 1 μm (2B), 4.5 μm (2C), 15 μm (2D), 20 μm (2E), 30 μm (2F), and 93 μm (2G).

FIGS. 3A-3E provide 2D arrays of PCL nanofibers produced in accordance with embodiments of the present disclosure. (3A) precisely aligned 2D arrays of nanofibers on the collection/supporting frame (inset); (3B) SEM image of single nanofiber with a 500 nm diameter. (3C-3E) SEM images of 2D aligned nanofibers with different periodicities: 200 μm, 100 μm, and 55 μm, respectively.

FIGS. 4A-4C demonstrate 3D scaffolding of PCL nanofibers: (4A) scheme of 3D scaffolding plan with spacer microfibers and nanofibers in two sets (See also FIGS. 7A-7B and 8A-8D). (4B) Optical image of two sets (See also FIGS. 7A-7B and 8A-8D) of nanofiber layers with 50±5 μm layer spacing arranged in an orthogonal orientation in 5×5 mm² area. Inset: fiber collection frame. (4C) Nanofibers aligned precisely with 100×100 μm² square lattice (SEM image). FIGS. 4D-4F provide Leica optical microscope images with the fiber collection frame in the inset (4D) and SEM images (4E, 4F) of aligned fibers at different angles, 30° and 60°, respectively.

FIGS. 5A-H illustrate 3D scaffolds that can be used for 3D cell culture according to embodiments of the present disclosure. FIG. 5A is an example of a 3D-scaffold on the collection frame with 8 sets (24 layers) of fibers consisting of 24 spacer fiber layers incorporated with 8 nanofiber layers alternatively crossed over each other. Inset: a macroscopic view of the collection frame with aligned fibers. FIG. 5B is an SEM imaging of 8-set scaffold in a bird-eye (or) corner view. FIGS. 5C-H are confocal microscopy images of RAW264.7 macrophages anchored to the nanofiber scaffold; (FIG. 5C) bright field; (FIG. 5D) z-stack of fluorescent images; FIGS. 5E, 5F show proliferation of macrophages on the nanofiber scaffold; FIG. 5G is a 110 μm z-stack fluorescence image taken with (FIG. 5F); FIG. 5H provides Imaris software analysis of z-stack presented in FIG. 5G.

FIG. 6 provides confocal microscopy images of RAW264.7 macrophages infiltrated into the interfiber space and anchored to fibers in accordance with embodiments of the present disclosure (nanofibers located in the space between microfibers are masked by the cells). Fibers are labeled with red fluorescein and cells are labeled with WGA. Fibers are observed in individual and mixed FITC channels (left to right).

FIGS. 7A-B illustrate nanofiber collection and alignment in accordance with embodiments of the present disclosure: FIG. 7A is one possible example of the setup; FIG. 7B is one possible example of a 3D scaffold with nanofibers and spacer fibers.

FIGS. 8A-D illustrate a possible embodiment of a fiber alignment setup in accordance with the present disclosure: (FIG. 8A) Perspective view focusing on the fixed and moving stages; (FIG. 8B) Left-hand side view focusing on the design of the fiber collector frame holder; (FIG. 8C) Front view focusing on a snapshot before starting the fiber collection; (FIG. 8D) Top view focusing on the groove and rotating fiber positioning arm.

FIG. 9 is a camera image of an example of a prototype nanofiber 3D alignment setup in accordance with embodiments of the present disclosure

FIGS. 10A-F illustrate a nanofiber collection and alignment process to develop 3D scaffold in accordance with embodiments of the present disclosure.

FIGS. 11A-D are optical microscope images of 2D aligned microfibers at different interfiber spacing: (FIG. 11A) 300 μm, (FIG. 11B) 200 μm, (FIG. 110) 100 μm, and (FIG. 11D) 50 μm.

FIGS. 12A-12D are diagrams of a 3D scaffolding plan for 2 sets of crossed fiber in accordance with embodiments of the present disclosure. (FIG. 12A) perspective view: 2 sets (FIGS. 7A-B and 8A-8D) of fibers with 45 μm gap between nanofibers. (FIG. 12B) top view of the frame with two sets of spacers and nanofibers. (FIG. 12C) Left (side) view. (FIG. 12D) Front (side) view: a is the distance between spacer fibers, A is the distance between nanofibers.

FIGS. 13A-130 are camera images of additive manufacturing of 3D-scaffold with 2 sets of crossed fibers in accordance with embodiments of the present disclosure: (FIG. 13A) Stage 1, spacer fiber scaffold in 3 crossed layers aligned in direction 1 and direction 2; (FIG. 13B) Stage 2, nanofibers aligned between the spacer fibers along direction 1; (FIG. 13C) Stage 3, nanofibers aligned between the spacer fibers along directions 1 and 2. The 2-set scaffold is fabricated by repeating steps 1-3 two times.

FIGS. 14A-14B provide optical microscope imaging of nanofibers aligned in 2 crossed sets. Both FIGS. 14A and 14B are images of the same area with different focus confirming the gap between nanofiber layers.

FIGS. 15A-15F illustrate cold drawing of GFD fibers: FIGS. 15A-15C are diagrams of the fiber drawn from a falling droplet with an initial length (lₒ=20 cm); FIG. 15B shows drawing/stretching the fiber to l₁=40 cm following the collection of a sample—an initial fiber section (orange color dotted square); FIG. 15C shows drawing the fiber to l₂=60 cm following the collection of the sample (as in case 15B). Optical images of the fibers are shown: FIG. 15D shows fibers collected in the situation illustrated in FIG. 15A, FIG. 15E shows fibers collected in the situation illustrated in FIG. 15B, FIG. 15F shows fibers collected in the situation illustrated in FIG. 15C.

FIG. 16 provides a possible embodiment of a spiral-winding device (not to scale) that can form sheet-like scaffolds that can be formed into various 3D shapes.

FIG. 17 provides another possible embodiment of a spiral-winding device, according to the present disclosure (not to scale). This device can be used for the fabrication of 3D tubular or cylindrical scaffolds.

FIGS. 18A-18D are examples of a device for spiral-winding of fibers according to embodiments of the present disclosure. 18A) corner view, 18B) Front view, 18C) supporting frame for fiber scaffolding. 18D) top view.

FIGS. 19A-19B provide an example of a spiral-winding device design (front view) with two steppers for holding the supporting frame according to embodiments of the present disclosure. 19A) tubular, cylindrical, etc. 19B) square, cube like structures etc.

FIGS. 20A-20D is one embodiment of a commercial-size 3D scaffolding device including a fiber storage wheel for batch processing design (20A, front view; 20B rear view; 20C, side view; 20D, top view), according to embodiments of the present disclosure.

FIGS. 21A-21D provide schematic representation of 3D fiber scaffolds (3DFS). Images are not scaled. 21A) top view. 21B) side view: stacked nanofiber arrays with defined spacing between layers. 21C) 3DFS cube geometry. 21D) a cage for controlled cell proliferation and free voids for media and gas exchange.

FIGS. 22A and 22B are examples of 3D fiber scaffolds according to embodiments of the present disclosure.

FIG. 23 is a plug and play 3DFS in a perfusion system vessel.

The drawings illustrate only example embodiments and are therefore not to be considered limiting of the scope described herein, as other equally effective embodiments are within the scope and spirit of this disclosure. The elements and features shown in the drawings are not necessarily drawn to scale, emphasis instead being placed upon clearly illustrating the principles of the embodiments. Additionally, certain dimensions may be exaggerated to help visually convey certain principles. In the drawings, similar reference numerals between figures designate like or corresponding, but not necessarily the same, elements.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, material science, and the like, which are within the skill of the art.

The following examples are put forth so as to provide those of ordinary skill in the art with a complete disclosure and description of how to perform the methods and use the devices and methods disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### Definitions

“Nanofiber” is a fiber having a radius on the nano-scale (e.g., 1 nm to 1000 nm).

“Polymer” is any natural or synthetic molecule that can form long molecular chains, such as nylons, polyethylene, polystyrene, polylactide, polyglycolide, polypropylene, polyacetylene, polyphenylene vinylene, polypyrrole, polyesters, polyurethanes, biopolymers including polycaprolactone, soy proteins, collagen, silk fibroin, combinations of these, and blends of these.

“Draw ratio” as described herein is a ratio of final length (L_{f}) after stretching the fiber to the initial length (Lᵢ) of the fiber after droplet fall, L_{f}/Lᵢ. Taking into account the conservation of volume (if a dry fiber is drawn) L_{f}/Lᵢ=(dᵢ/d_{f})\hat{ }2.

### General Discussion

In accordance with the purpose(s) of the present disclosure, as embodied and broadly described herein, embodiments of the present disclosure, in some aspects, relate to methods of making nanofibers, methods of making 2D nanofiber arrays and 3D nanofiber scaffolds, and devices for making nanofibers, 2D nanofiber arrays, and 3D nanofiber scaffolds.

In general, embodiments of the present disclosure provide for devices and methods for making nanofibers and scaffolds that include nanofibers made by gravitational drawing.

An embodiment of the present disclosure includes method for making single filament nanofibers. Advantageously, the fiber can have submicron diameter and can be tuned to have desired properties. The nanofibers are formed using gravity to draw the fibers, which allows for fiber drawing without the need for materials with magnetic properties or electrospinning.

Embodiments of the present disclosure include a method as above, wherein a single nanofiber can be formed by dispensing a droplet of polymer solution from a nozzle onto a base. The nozzle (e.g. a nozzle, syringe, or needle) is positioned above the base such that the droplet can be pulled by gravity to free fall from the nozzle onto the base. The falling droplet of polymer solution is drawn into a fluid tail between the base and the nozzle. As the fluid tail is being drawn, solvent in the polymer solution evaporates, causing the fluid tail to solidify to form a nanofiber. The nanofiber can have a diameter of about 50 nm-100 μm and a length of about 1 cm to 10 m, or about 10 cm to 2 m. The fiber diameter and the fiber length can be tuned by adjusting the distance between the nozzle and the base. Advantageously, these fibers can be used alone, for twisting or weaving, or organized in 3D scaffolds as described below.

The methods and devices of the present disclosure allow for formations of nanofibers and nanofiber scaffolds that do not rely on the dielectrical or magnetic properties of the nanofiber-forming material, using instead simple gravitational force applied to materials with a high draw ratio, such as the draw ratios known for the synthetic and natural polymers listed herein. Most synthetic and natural polymers can be used to fabricate fibers by gravitational drawing in their melt state, or solution state. The polymers can include, but are not limited to, such as polyethylene oxide, polycaprolactone, polyacrylonitrile, polystyrene, polyvinyl acetate, polylactic acid, Teflon (copolymer of 2,2-bis(trifluoromethyl)-4,5-difluoro-1,3-dioxole), poly(3-hexylthiophene), MEHPPV (poly[2-methoxy-5-(2-ethylhexyloxy)-1,4-phenylenevinylene]), PVDF (polyvinylidene fluoride) and its copolymers, pectin, soy, alginate, chitosan, polyolefin, polyesters, cellulose, protein (e.g. silk fibroin). In the solution state, the polymers can be dissolved in their respective solvents, such as benzene, DMF, acetone, DMAc, ethanol, water, chloroform, N-methylmorpholine oxide, Fluorinert FC-40, toluene, tetrahydrofuran, and carboxylic acid, or combinations thereof. Other FDA approved polymers or polymer blends can be used as well.

Polymers and polymer blends and suitable solvents used to form polymer solutions can be optimized for particular cell types or desired applications. The gravity fiber drawing methods described herein utilize optimization of solvent or solvent mixtures to prepare polymer solutions using single polymer or blends of polymers. The choice of aqueous and/or organic solvent mixtures to prepare each polymer solution varies. For instance, polycaprolactone in only chloroform as solvent can be suitable to draw microfibers of about 2 μm in diameter, and chloroform:benzene mixture in 1:1 ratio can be used to draw fibers of submicron diameter. In one example, polymer blends of polycaprolactone with PGMA [poly(glycidyl methacrylate)] of different ratios is beneficial for 3D cell culture applications to improve protein coating and cell adhesion. The epoxide groups in PGMA allows functionalization of the fibers with cell specific chemical cues post scaffolding prior to 3D cell culture. Ability to modify the surface of fibers to suit different cell types—enables the scaffolds of the present disclosure to be used as a near universal tool (e.g. 2D cell culture plates) for practically any cell type. For example, synthetic and nature polymer blends can be used in tissue engineering.

In various embodiments, the fiber diameter and other fiber properties such as fiber storage modulus and draw ratio can be tuned based on parameters of the polymer solution such as density, viscosity, surface tension, and the volumetric evaporation rate per unit area of the solution. Desired fiber properties, such as ducility, stretchability, conductivity, flexibility, or others can be engineered based on the polymer solution and device set-up used. In various embodiments, the polymer solution includes about 5 to 15 wt % polymer. In some embodiments, the polymer can be a biopolymer and can be dissolved in an aqueous solution. The solution can, depending upon the molecular weight of the polymer, include about 5 to 15 wt %, about 15 to 20 wt %, or greater than 20 wt % polymer. In some embodiments, the polymer solution can include about 5 to 15 wt % poly-ε-caprolactone. In some embodiments, the polymer solution can be a mixture of different synthetic and biopolymers (e.g. polyethylene oxide with PCL, PCL with polyglycidyl methacylate, collagen and PCL), and other combinations as can be envisioned by one of ordinary skill in the art. In some embodiments, the polymer solution includes chloroform, benzene, or both as a solvent. In some embodiments, the solvent is a non-polar solvent. In some embodiments, the polymer solution can be polyethylene oxide in 1,4-dioxane, polyethylene oxide in acetonitrile, or polyethylene oxide in methanol. In various embodiments, the polymer solution can include a surfactant such as Triton. Other solvent or solvent mixtures can be used depending upon the polymer or polymer blends and one can appropriately select the solvent or solvent mixture based on the polymer or polymer blend used. The diameter of the nanofiber can be adjusted by varying surface tension of polymer solutions, vapor pressure of the solvent, concentration of the polymer and distance between the nozzle and base. In some embodiments, the nozzle diameter can also be changed to further control the fiber diameter and length.

The present disclosure provides for methods of forming nanofiber scaffolds. A first single filament nanofiber is formed by gravitational drawing of a polymer solution from a nozzle to a base. The polymer solution includes a polymer and a solvent; while the drawing occurs, evaporation of the solvent causes the polymer solution to solidify into a fiber. The first single filament nanofiber can have a diameter of about 50 nm-100 μm. The first single filament nanofiber can then be collected on a collection frame. The process is repeated so that one or more subsequent single filament nanofibers are formed and collected on the collection frame, thereby forming a first 2D array layer. The layer can be oriented in a plane, e.g. in an X-Y plane. The properties of each subsequent nanofiber, such as position, thickness, or polymer, can be independently selected.

Each nanofiber can be spaced and aligned in a predetermined, non-random manner, such that the resulting array of nanofibers is ordered and precisely spaced according to a particular need. For example, the distance between a pair of nanofibers is at a set distance (e.g., 10 μm to 100 μm, 10 μm to 20 μm) and/or at a desired angle (e.g., 0 to 179 degrees) relative to each other or to another fiber in the array. The fibers can be parallel to one another and/or in any desired angle and position relative to the first single filament nanofiber. In some embodiments, all of the nanofibers in a given array will all be parallel to one another. In some embodiments, the fibers can be placed at predetermined angles relative to one another or to the collection frame. In some embodiments, the fibers can be collected (e.g. non-randomly placed on the collection frame) such that they form crisscross or woven patterns in a given array.

Advantageously, the methods and devices described herein can produce precisely aligned nanofibers having controlled spacing and orientations that cannot be achieved using rotating fiber drawing methods (e.g. electrospinning, magnetospinning, touch spinning, etc.). Nanofiber formation methods such as those described above can be used to form ordered arrays of nanofibers. The arrays can then be layered to form three-dimensional scaffolds that can be customized with varying fiber materials, diameters, elastic modulus, orientations, angles, spacing, spaces between layers, and geometries (e.g. flat sheet, tube, cylinder, cube, etc.). Multilayer scaffolds with this level of tunability are not possible using other fiber drawing methods such as solution electrospinning due to liquid jet instabilities and spatial limitations because the filaments drawn between the nozzle and the electrode cross the space occupied by previously deposited fibers.

In various embodiments, multiple layers of 2D arrays can be formed into a 3D scaffold in an additive fabrication approach, and the layers can optionally be separated by spacer fibers. These spacer fibers can be formed using similar methods as the nanofibers. The spacer fibers can be formed from the same or different polymer solution from the nanofibers. The spacer fibers can be deposited from the same or a different nozzle than the nanofibers. Spacer fibers can be nanofibers or microfibers. In some embodiments, the spacer fibers can be about 1 μm to 100 μm in diameter. In various embodiments, the spacer fibers can comprise soluble or insoluble polymers. Soluble polymers, such as polyethylene oxide and polyvinyl alcohol, can be used when it is desirable for the spacer fibers to be sacrificial (e.g. not needed after formation of the 3D scaffold). Insoluble polymers, such as polystyrene or polypropylene, can be used when the spacer fibers will not be removed after formation of the 3D scaffold. In some embodiments, the spacer fibers can be formed separately from the nanofibers (e.g. by 3D printing) and introduced as a layer between nanofiber array layers. In other embodiments, the spacer fibers can be formed using the nanofiber forming devices or methods as described herein.

In some embodiments, at least two spacer fibers can be deposited on a 2D array. In some embodiments, more than one spacer fiber can be stacked to form a thicker layer. Multiple layers of spacer fibers can be laid between nanofiber array layers. The orientation of the spacer fibers can be the same as the nanofibers, can be placed orthogonally, or placed at another angle.

A second 2D array layer can be formed as described above and stacked on the spacer fiber layer to form a 3D scaffold. For example, a 2D nanofiber array forms a first layer having nanofibers in the x-y plane, followed by a spacer layer having fibers in the x-y plane, followed by a second 2D array layer having nanofibers in the X-Y plane. By alternately forming and collecting additional spacer fiber layers and 2D array layers in the X-Y plane, a 3D scaffold extending in the z-direction is formed. While the nanofibers and spacer fibers are all oriented in an x-y plane, each spacer fiber layer or 2D array layer can be rotated in the X-Y plane relative to a fiber orientation in the first 2D array layer. In this way, complicated, tunable patterns can be formed beyond parallel and orthogonal fiber layers.

In various embodiments, each nanofiber can independently have a diameter of about 50 nm-100 μm. In various embodiments, the distance between any two nanofibers in a 2D array layer can be about 10 μm or greater, or about 10 μm to 1000 μm. In some embodiments, the distance between any two nanofibers in a 2D array layer can be more than 1000 μm. In various embodiments, the fiber orientation between any two layers of fibers can be between 0° and 179°. In various embodiments, the fiber orientation between any two fibers within a layer can be between 0° and 179°. Depending upon the desired properties of the scaffold (e.g. mechanical strength or ability to allow cell infiltration) individual nanofibers in the scaffold can have the same or different diameters from one another, and individual nanofibers can have the same or different compositions.

Advantageously, the porosity of the layers and scaffolds can be tuned to meet specific purposes. In various embodiments, the scaffold can have a porosity of about 20% to 99%, about 50% to 99%, or about 90% to 99%.

In some embodiments, the spacer fibers can be removed or dissolved once the 3D scaffold has been formed. Such sacrificial fibers can be used in scaffold geometries where large inner spaces are desired, such as tubes or cylinders. Sacrificial spacer fibers can also be useful in scaffolds used to mimic complex vascular tissue or scaffolds used for 3D cell culturing.

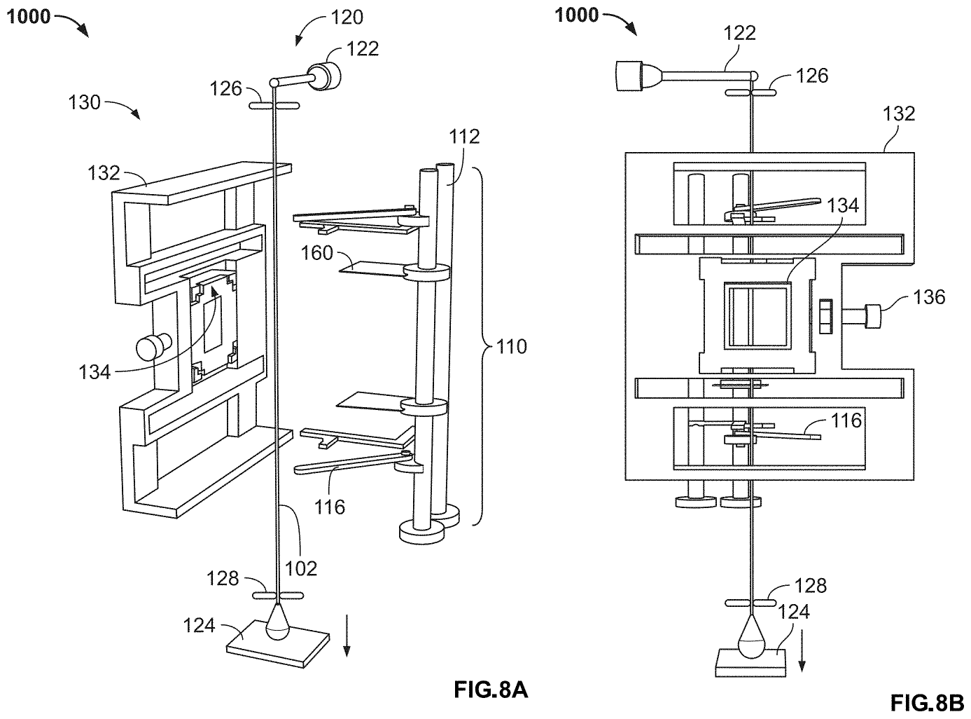

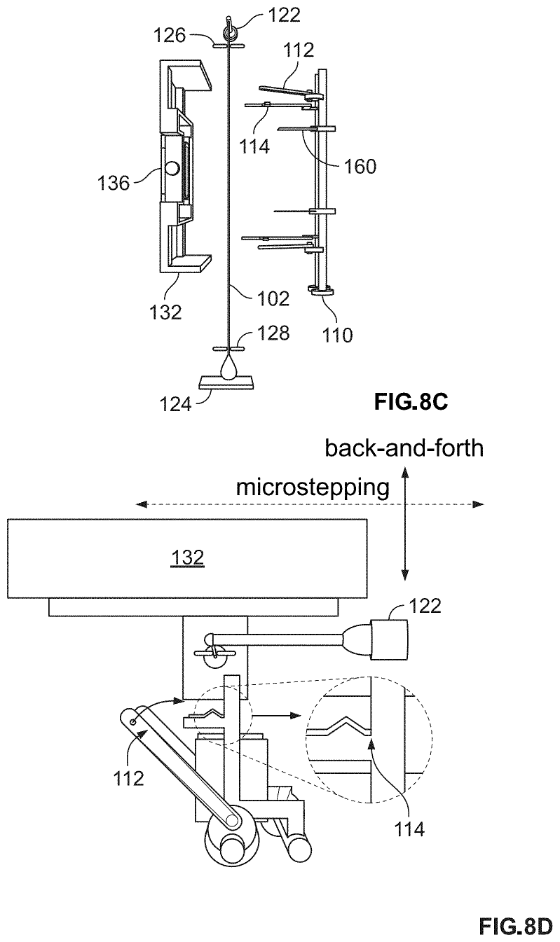

The present disclosure also provides for devices for forming nanofiber scaffolds. The device can include a fiber positioning assembly, a fiber drawing assembly including a nozzle positioned above a tension stage, a fiber collection and delivery assembly including a moving stage, and a cutting tool. In various embodiments, the fiber positioning assembly comprises includes positioning arms. Grooves for holding fibers in place during clipping or placing can also be included (see FIG. 8D). The fiber positioning assembly can include one or more vertical poles, upon which the fiber positioning arms can be affixed. In some embodiments, the fiber positioning arms can be affixed substantially perpendicularly to the pole and can move laterally (e.g. swing or flip) to help position the fibers. The poles can also include an arm or arms containing grooves or clips as described above. The positioning arms can swing to place the fibers in the grooves. In some embodiments, the arm or arms with grooves can be on a first pole, and the fiber positioning arms can be on a second pole.

In various embodiments, the device includes fiber anchors. The fiber anchors can be moveable or fixed. The fiber anchors hold fibers for positioning, cutting, collection, and delivery. The fiber anchors can be included in various locations on the device. For example, fiber anchors can be included on the fiber positioning assembly, the fiber drawing assembly, the fiber collection and delivery assembly, or the fiber scaffolding assembly. The fiber anchors can be simple clips, hooks, or grooves. The anchors can be placed on an assembly to anchor the top and bottom of a fiber into place. In some embodiments, the fiber anchor can be combined with a fiber cutting mechanism to form a hybrid fiber anchor, such that the anchor can hold and then cut the fiber.

In various embodiments, one or more fiber cutting mechanisms (such as blades, hot blades, or hot wire filaments) can also be included. The fiber cutting mechanism can be included on the fiber positioning assembly. In some embodiments, the fiber cutting mechanism can be included on the fiber collection and delivery assembly. In yet other embodiments, the fiber cutting mechanism can be included on the fiber drawing assembly. Depending on the placement of the fiber cutting mechanism, the fibers can be cut at various stages of the fiber scaffold formation. For example, the fibers can be cut after drawing but prior to positioning, cut during positioning but before collecting, or cut after collection once the layer or scaffold has been formed.

In various embodiments, the fiber collection and delivery assembly can include a fiber collector (also referred to as a moving stage) that can be rotated about an axis (e.g. the X-axis). In some embodiments, the fiber collector can include a fiber collector frame. In various embodiments, the moving stage can move along an axis (e.g. the X-axis) to collect parallel nanofibers to form a 2D array on the fiber collector frame. The moving stage can also move along an axis (e.g. the Z-axis) to form a 3D scaffold from layers of 2D arrays. In some embodiments, the fiber collector frame can be rotated within the moving stage to allow for precise placement angles of individual fibers.

In various embodiments, a fiber scaffolding assembly can also be included. The fiber collection and delivery system can be integrated into the drawing assembly. The addition of the fiber scaffolding assembly allows for fiber winding, such that the 3D scaffold can have a 3D shape not limited to a rectangle. The fiber scaffolding assembly can include at least one stepper motor. The stepper motor can drive linear motion of the fiber scaffolding assembly and rotational motion of the fiber collector. In this manner, the fibers can be coiled around the frame, also described herein as spiral winding. 2D arrays of precision aligned nanofibers and 3D scaffolding on any supporting frame can be achieved with collections of appropriately cut individual fibers in sequence or by coiling a single, long (e.g. about a meter) fiber around the supporting frame. In an embodiment, the supporting frame is a rectangle, and when the fiber is coiled around the supporting frame, a nanofiber array is formed on two sides of the supporting frame. In various embodiments, the supporting frame can be selected from 2D or 3D shapes, including but not limited to rectangles, cubes, prisms, cylinders, ovoids, triangles, pyramids, polygons, or irregular shapes. Instead of placing single fibers in a sequence to create an aligned 2D layer of nanofibers, the supporting frame can be rapidly coiled a single long nanofiber to obtain aligned nanofibers.

In various embodiments, the supporting frame can be comprised of a soluble material. The soluble material can be dissolved after formation of the fiber scaffold, leaving only the fibrous scaffold. In other embodiments, the supporting frame can be made from polymer, glass, metal, ceramic, etc.

In various embodiments, the fiber positioning assembly comprises one or more fiber storage and delivery wheel. The fibers are collected on arms attached to the wheel having fiber anchors. As the wheel rotates, one fiber is collected as another is delivered to a fiber collection and delivery assembly for cutting and transfer to a 3D scaffolding assembly.

In various embodiments, more than one nozzle can be included. Advantageously, multiple types of fibers can be drawn from different nozzles dispensing different polymer solutions, or fibers having different diameters can be drawn from nozzles of different sizes. For example, fibers for the scaffold can be drawn from a different nozzle than the spacer fibers. In some embodiments, the nozzles can be included together in a cartridge. In some embodiments multiple nozzles can be included to draw fibers for collection on individual arms of a storage and delivery wheel as described above. In some embodiments, the nozzles are not positioned together, depending on the configuration of the device and the desired characteristics for a particular scaffold. In other embodiments, the device can be configured to include more than one fiber drawing assembly, each of which is associated with a fiber collection and delivery assembly. In this manner, fibers could be delivered to both the front and back sides of a support frame, for example.

In various embodiments, the devices described herein can include controllers to allow programming and automation of the fiber scaffold process. A user can feed the polymer solution or solutions to the fiber drawing assembly, similar to a 3D printer, and the device will produce a fiber scaffold according to parameters input by the user.

In some embodiments, the polymer solution-based spacer fibers could be replaced with melt drawing. This may be desirable in some applications, such as where large diameter fibers are required for spacers in the 3D scaffolds. For example, melt drawing can be utilized if the scaffold sizes are larger than about 20×20 cm² to provide a more uniform diameter along the length of longer spacer fibers.

The methods and devices described herein can be used to form 3D cell culture scaffolds. Such scaffolds can be formed with specially designed gaps to allow for nutrient, gas and metabolite exchange. In one embodiment, the gaps can be about 200 μm between multilayered fibers of 200 μm thickness). This type of cell culture scaffold can reduce necrosis in 3D cell aggregate and help the continuous growth of culture for weeks. Advantageously, commercially interesting metabolites (e.g. antibodies, exosomes, etc.) can be extracted efficiently. 3D cell culture scaffolds as described herein can be used to scale up the 3D cell culture techniques for industrial scale metabolite or cell-based therapeutics production as an alternative to bioreactors. In existing 3D cell culture methods, the mechanical properties of fibers cannot be controlled. The gravitational fiber drawing (GFD) methods described herein can be used to stretch/draw the hanging fiber with a controlled rate and distance to modulate the crystallinity in the fiber, thus tuning its mechanical properties.

The present disclosure provides for 3D cell culture scaffolds that include at least two arrays of fibers formed according to the methods described above. The fibers can have diameters between about 50 nm and 2 μm. The fibers can include a biocompatible polymer suitable for using as is or surface functionalization with proteins or biochemical cues post scaffolding. In various embodiments, the scaffold can have a porosity of about 50% to 99%, about 70% to 99%, or about 90% to 99%.

Key issues with existing 3D cell culture technologies include poor nutrient, gas, and metabolite exchange between the extracellular matrix and external environment with culture media. This can result in 3D cell necrosis causing workflow problems and costing time and resources. Advantageously, the 3D cell culture scaffolds described herein can be formed with over 90% free volume (e.g. porosity) and uninterrupted predesigned channels that allow for nutrient, gas and metabolite exchange until the 3D cell culture is harvested. The nanofiber spacing and alignment of each fiber can be precisely controlled during manufacture. In various embodiments, the 3D cell culture scaffolds can be formed on frames of about 1×1 cm²-15×15 cm² dimensions for laboratory scale research applications, which allow the scaffolds to fit in standard cell culture flasks. Further, the 3D fiber scaffolded frames could be combined with a cup-like insert to fit into standard multi-well cell culture plates, or other insert shapes to fit into petri dishes and T-flasks for cell culture. Scaffolds larger than 15×15 cm² are suitable for commercial size scale-up needs and can be designed as stack of scaffold frames to fit into bioreactor style containers with continuous circulation of all exchange materials (e.g. nutrients, gas, metabolites, etc.). The applications of these scaffolds include but are not limited to 3D cell cultures and tissue engineering.

In some embodiments, the arrays comprise high-density fiber sections and low-density fiber sections, such that the layered arrays form cell growth areas and media transport areas in the 3D scaffold. In some embodiments, multiple scaffolds can be stacked in multiple directions, e.g. like bricks or towers, to form larger 3D scaffolds without sacrificing stability.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

Methods for drawing single filament nanofibers from polymer solutions and employing them as building blocks to fabricate precisely aligned 3D structured scaffolds are highly demanded for mimicking microstructure of extracellular matrices (ECM) for growing 3D cell cultures and complex tissues in biotechnology and regenerative medicine applications.^{[1]} Described herein is a gravitational fiber drawing (GFD) method which produces monofilament nano- and microfibers (about 50 nm-100 μm in diameter) and enables assembling them in highly ordered 3D arrays with controllable interfiber spacing (about 10-1000 μm) and fiber angular orientation (between 0° and 360°). GFD can be utilized to produce complex 3D constructs made of fibers with different diameters, draw ratios, compositions, and organizations via an additive manufacturing approach.

Research and applications of micro- and nanofiber are currently in a phase of continuing growth^{[2]} that is evidenced by numerous examples of their applications in nanofilters,^{[3]} protective clothing,^{[4]} wound dressings,^{[5}] 3D cell cultures,^{[6]} scaffolds for tissue regeneration and drug delivery,^{[7]} electrode materials in batteries and fuel cells,^{[8]} sensors, microelectromechanical systems, flexible electronics,^{[9]} anisotropic optical materials,^{[10]} and nanofluidic structures,^{[11]} etc.

Well-organized nanofibers with precisely controlled interfiber spacing in all 3D dimensions are essential attributes of 3D anisotropic nanomaterials. Macroscopically long monofilament nanofibers properly organized in 3D-structures is one of the possible solutions of a long-standing problem of making nanostructured material across multiple length scales from nano- to macroscopic dimensions. Optimized interfiber spacing is critical for many applications that rely on mass transport of ingredients from tissue engineering to filtration and energy storage. Fibrous mats with randomly oriented nanofiber networks and a broad pore size distribution are less efficient for precise filtration, electrical, optical devices, and mimicking natural fibrous scaffolds for tissue regeneration applications. Obviously, a combination of a controlled nanofiber diameter, mechanical characteristics, and high degree of organization (spacing and orientation) shall be used to tailor biological, electrical, optical, and mechanical properties of fibrous 3D nanomaterials.

Several broadly explored methods for nanofiber alignment use the drawing of fibers in an electrical field (electrospinning, ES), by centrifugal, and mechanical forces.^{[9a, 12]} In a widely used ES technique, the nozzle is subjected to a high voltage. The collector is connected to the ground. Nanofibers with anisotropic orientation are easily fabricated and collected on the collector electrode (pan or mandrel) by drawing the thread of the polymer solution or melt ejected through the nozzle and winding the formed fiber about a rotating mandrel-electrode.^{[13]} Despite its simplicity, efficiency, and scalability, the conventional ES and other rotating spinneret methods are not suitable to fabricate precisely aligned nanofibers into 3D scaffolds with controlled spacing and orientation because of instabilities of liquid jet.

The effect of instabilities could be minimized by minimizing the distance between the injecting (e.g., nozzle) and fiber collecting (e.g., electrode) modules to as small as few millimeters. This approach was realized in different variants of ES, such as a melt electrospinning (MES),^{[14]} and near field electrospinning (NFES) techniques^{[15]} These methods demonstrated substantial improvement in fiber spacing and alignment as well as 3D scaffolding capability. MES and NFES methods can be automated if combined with 3D printing technology. However, at such a small drawing distance, the drawing process has limitations for adjustment of draw ratio, fiber diameter, and formation of highly porous 3D-constructs. The fiber diameter with MES is restricted to several tens of microns. Currently, NFES is the only promising technique to fabricate nanofiber scaffolds with controlled fiber spacing and organization. NFES has yet limitations to generate multilayer scaffolds since filaments drawn between the nozzle and the electrode are crossing the space occupied by previously deposited fibers. Another limitation of a relatively small drawing distance results in a very short time for solvent evaporation and, thus, may potentially destroy the structure by residual solvent, fusing layers and providing poor control over draw ratio and hence, the mechanical properties of the fibers.^{[15a]}

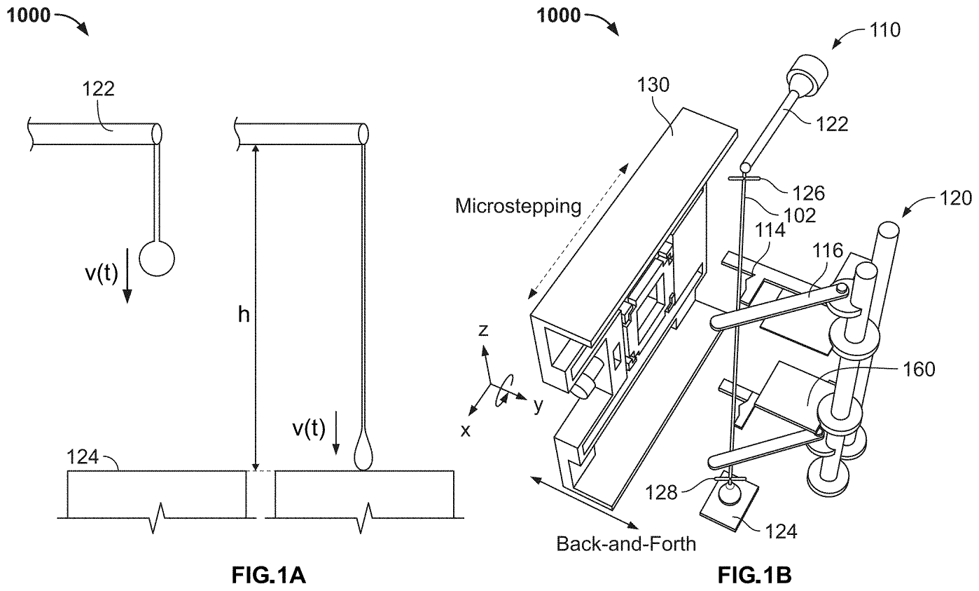

Described herein is a simple GFD technique when a polymer solution (5 to 15 wt %) is pumped through a nozzle 122 (e.g., syringe, needle) followed by a free fall of the droplet. Turning to FIGS. 1A and 1B, the falling droplet generates a meters long single filament nanofiber 102 hanging between the nozzle tip, and the droplet lands on the surface of the base 124 (FIG. 1A). As shown in FIG. 1A, a droplet of the fluid falls from a syringe nozzle 122 located at a height h above the ground level. As the droplet falls, it draws out a thin fluid thread behind. The solvent evaporates, and the fluid solidifies to form a fiber. The fiber diameter is governed by the droplet falling distance, polymer solution surface tension, density, viscosity, and the volumetric evaporation rate per unit area of fluid stream surface, which further depends on the cross-sectional area exposed to air. By keeping the parameters fixed and varying the droplet falling distance, the fiber diameter and its variation along the filament length are well reproduced.

GFD offers the most straightforward approach to fabricate several meters in length single monofilament fibers of a submicron diameter. These macroscopically long nanofibers can be isolated and used as building blocks in a bottom-up microfabrication approach for constructing a complex 3D scaffold with controlled spacing (A) and alignment angle (θ) by their precise deposition. The freestanding fibers could be used for twisting and weaving on demand.

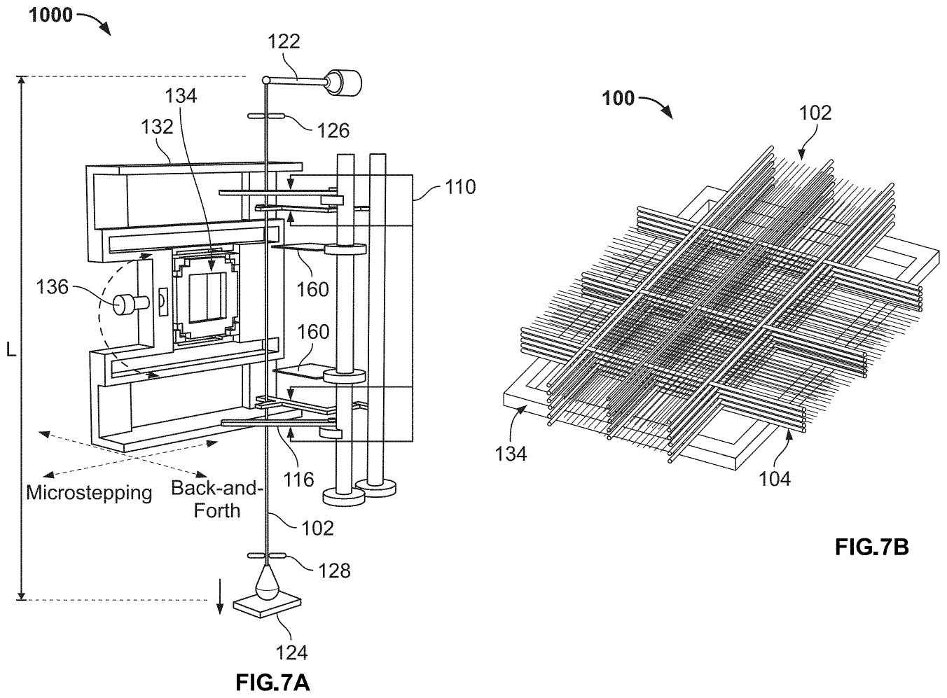

The fabrication of 3D scaffolds using the GFD method includes collecting individual nanofibers, depositing them in a 2D array, which is followed by adding step-by-step other layers with desired dimensions. The multistep process can be automated, as shown in FIGS. 1B, 7A-B, 8A-D. The schematic shows a device that is only a variant of many possible designs for single nanofiber handling and scaffolding using the GFD method. It is comprised of three main parts: a fiber drawing assembly 110, a fiber positioning and scaffolding assembly 130, and a fiber collection and delivery assembly 120 working in sequence to handle individual nanofibers and align them on a scaffold supporting frame. Further description of the scaffolding mechanical device and fiber collection process is provided in Example 2.

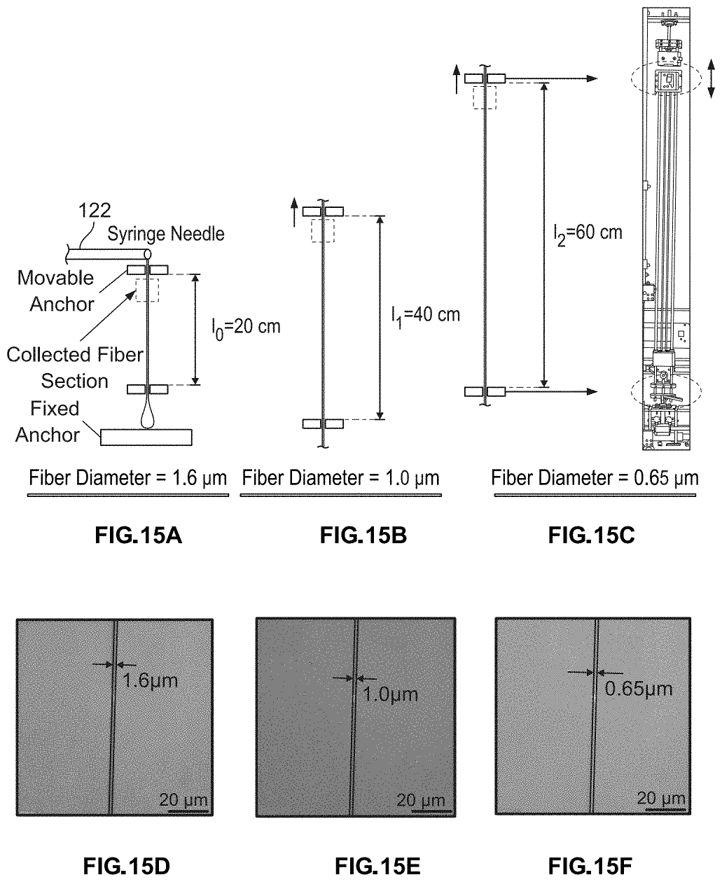

The dry fibers can be cold/hot drawn, for example drawn from a hot polymer solution or the fabricated fiber can be drawn/stretched as in FIG. 15A-15F. Tensile stretching induces molecular level polymer chain alignment, which induces crystallization and enhances mechanical properties. The tunability of the nanofiber draw ratio is a very interesting feature offering the possibility to regulate mechanical characteristics of the nanofibers with no changes in their chemical composition.

TABLE 1
Cold drawing of single filament fiber (di is the initial fiber diameter,
df is the final (after drawing) fiber diameter). The initial length is 20 cm.

| collected fiber sample from a single filament | fiber diameter, [μm] | $\begin{matrix}draw ratio \\ {( \frac{di}{df} )}^{2}\end{matrix}$ |
|---|---|---|
| 1 (no stretch) | 1.6 | — |
| 2 (1^{st} draw) | 1.0 | 2.6 |
| 3 (2^{nd} draw) | 0.65 | 6 |
| 4 (3^{rd} draw) | 0.5 | 10.2 |

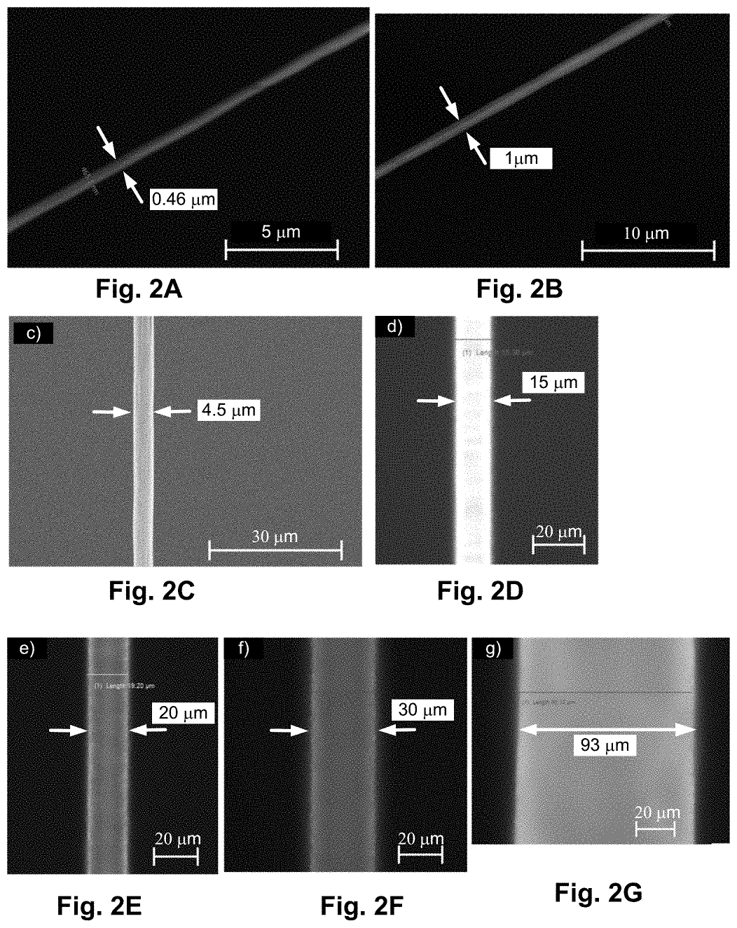

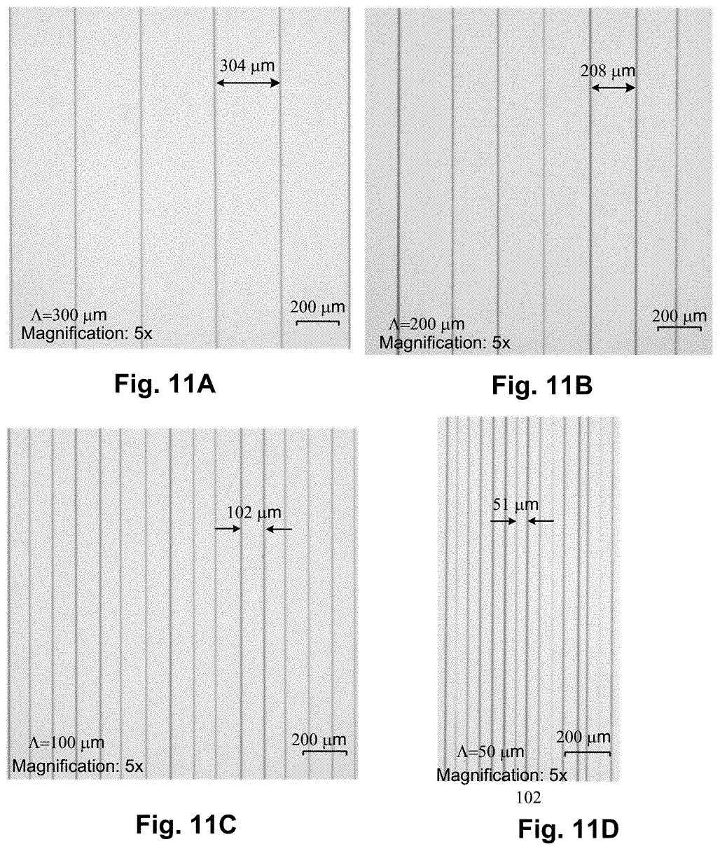

An example of poly-ε-caprolactone (PCL) nanofibers demonstrates the capability of the GFD technique. The surface morphology of the gravitation drawn fibers is smooth (FIG. 2A-2G), similar to those made by other methods of nanofiber fabrication.^{[12b]} Submicron PCL fibers were drawn from solutions made of 1:1 molar mixture of chloroform and benzene solvents. The PCL fiber of a larger diameter (1 μm-100 μm) is easily controlled by adjusting the PCL concentration in chloroform and droplet falling distance. SEM and optical microscope images of nano and microfibers are presented in FIGS. 2A-2G and 11A-D. Microfibers (>5 μm) are typically fabricated by a PCL melt-spinning method. However, the fabrication of fibers in the broad range of dimensions from nanometers to microns by the one device is beneficial for the fabrication of complex structure scaffolds. Also, not all functional additives (e.g., proteins) could withstand high temperatures of melting. Variations of the draw ratio are demonstrated in Table 1 and Example 2.

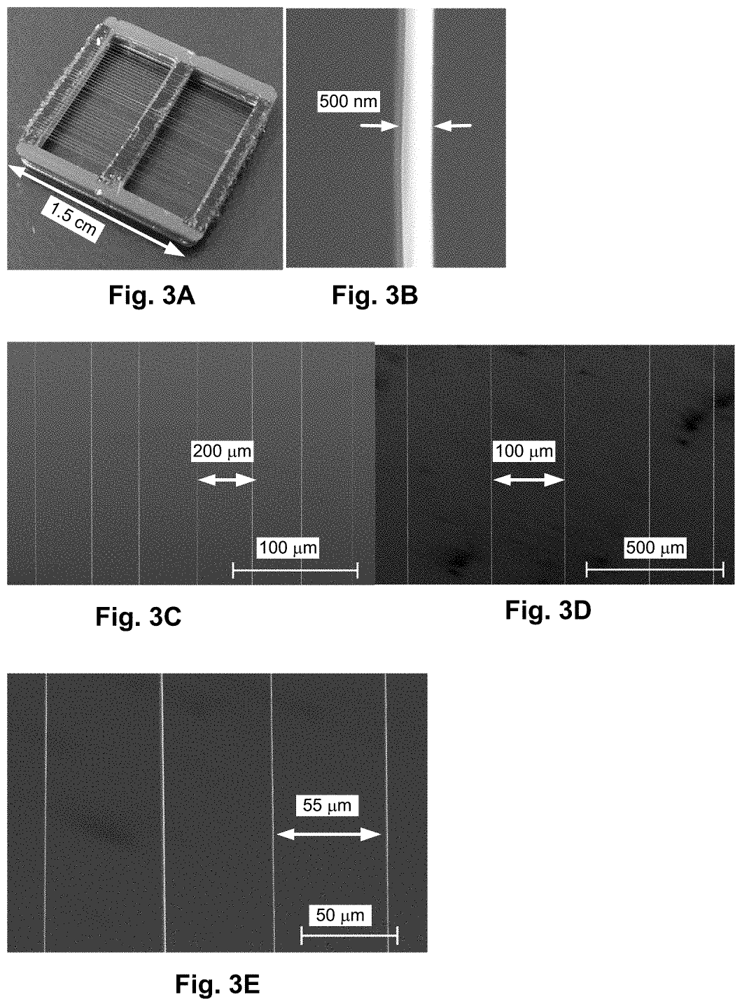

Fabrication of a 2D array of nanofibers with a precise spacing of 50 μm between the collected fibers attached to the collection frame can be realized by moving the moving stage with a 50 μm step each time after the fiber formation by the falling droplet. The 2D array is formed in a sequence with the repetition of the fiber drawing and collection process until the sample size is achieved. Examples of precisely aligned 2D arrays of nanofibers with A=200±10 μm, 100±10 μm, and 50±5 μm are shown in FIGS. 3C-3E, respectively. The reproducibility of ˜95% is observed in the fiber collection process, with only one or two fibers missing in the sequence. Other alignments and spacing can be achieved, as can be envisioned by one of skill in the art.

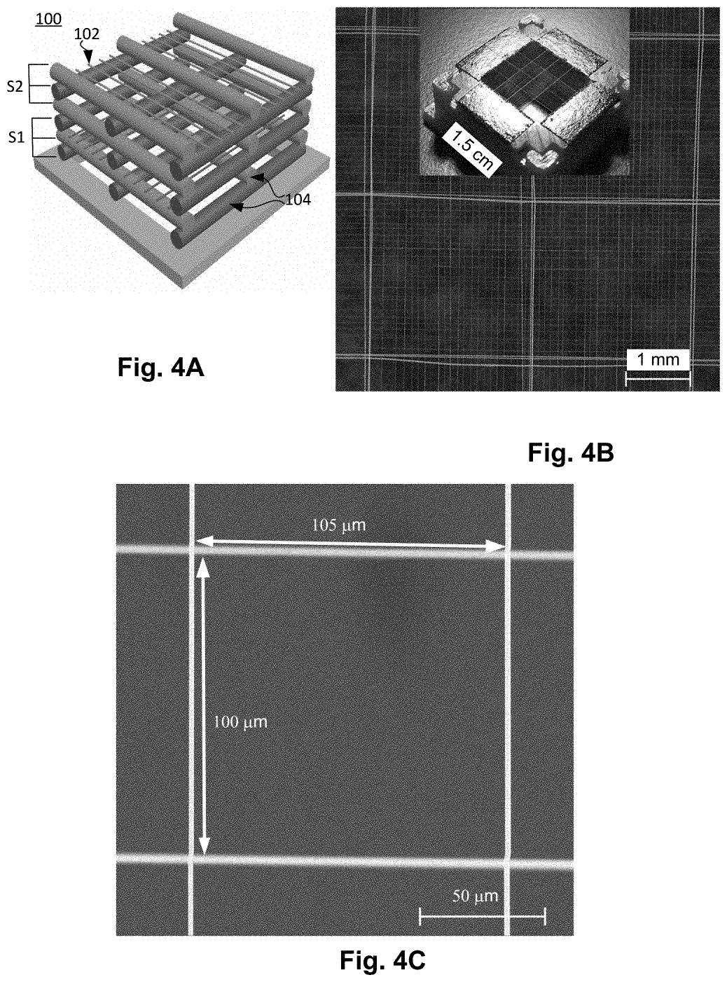

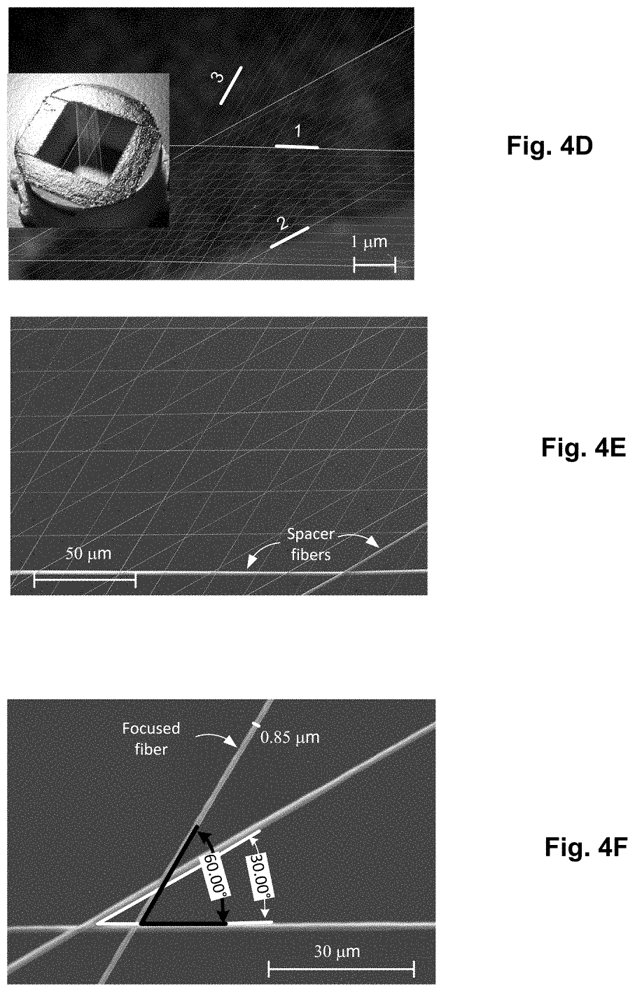

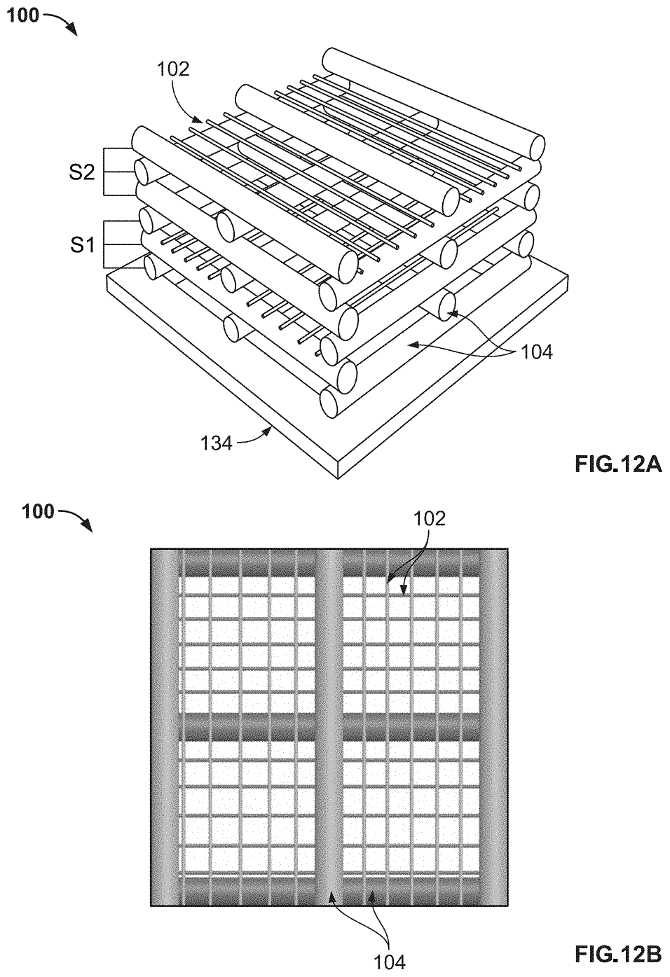

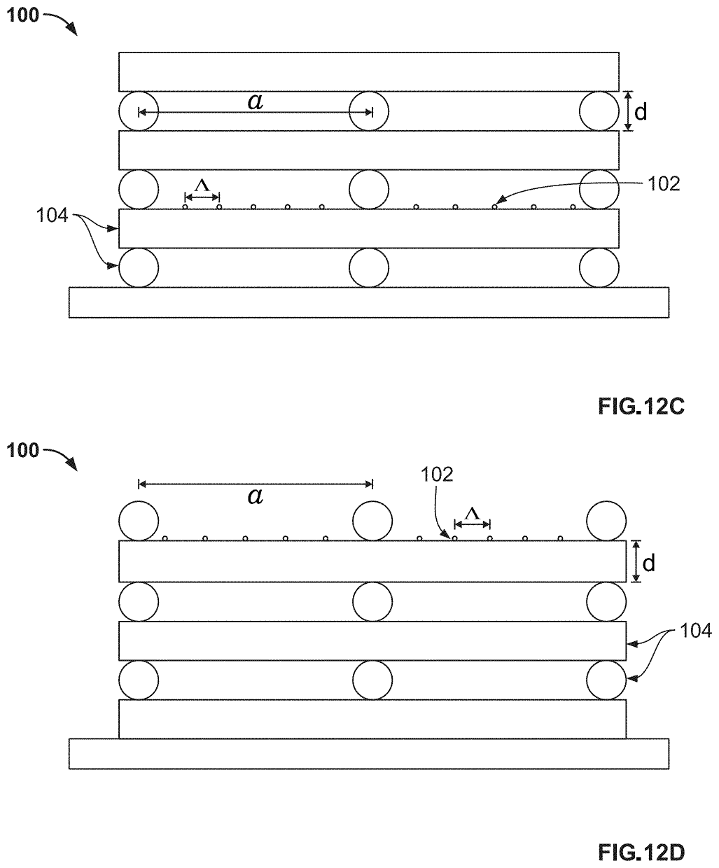

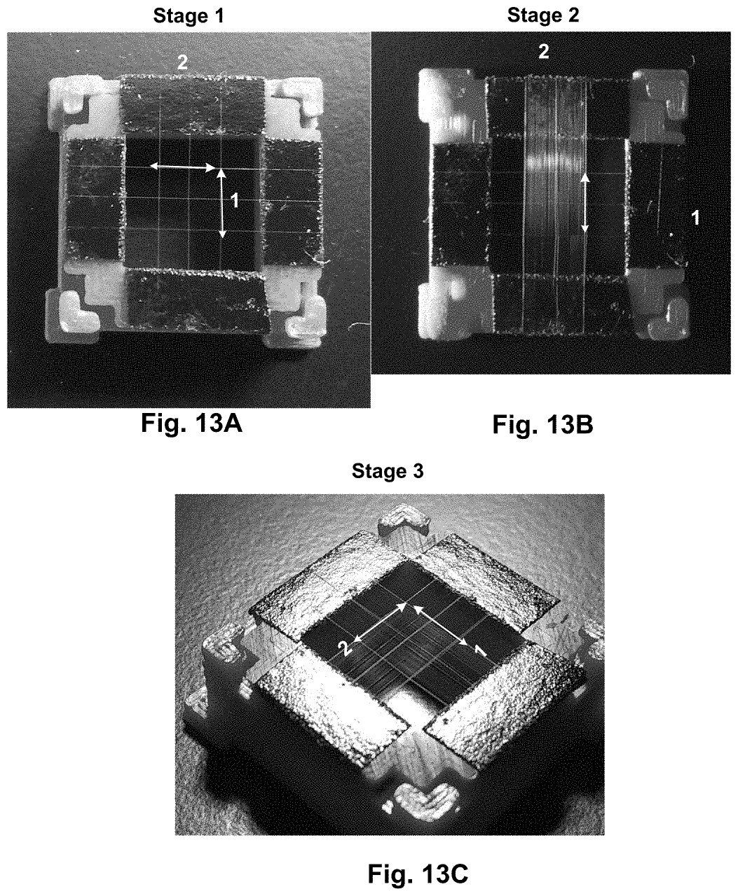

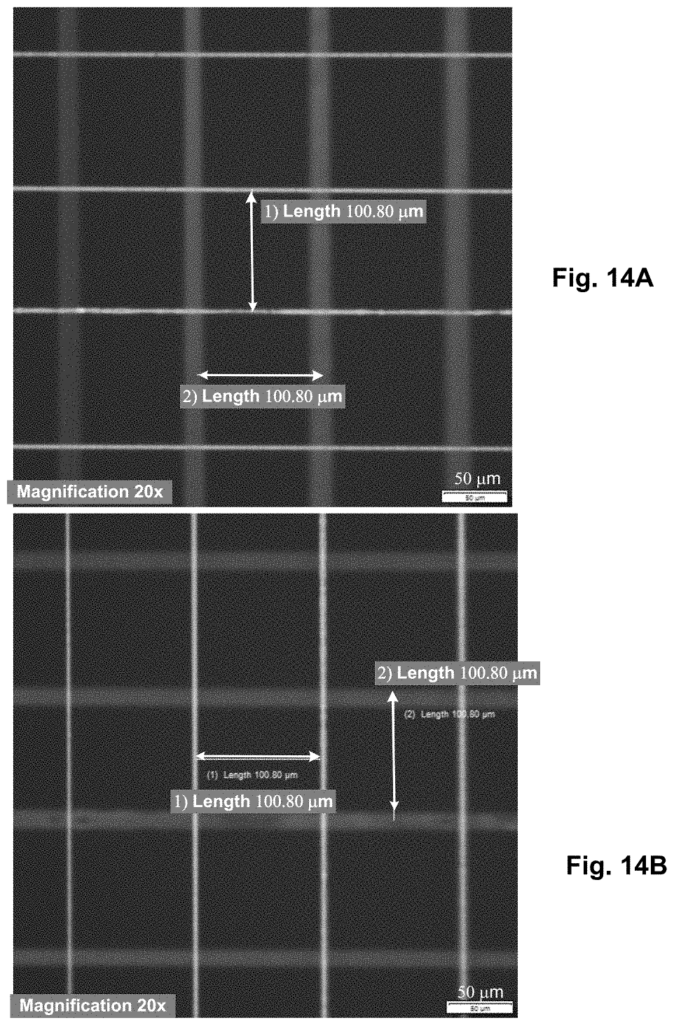

This method for fabrication of 2D array can be further extended to 3D scaffolding 100 using a simple scaffolding plan presented in FIG. 4A and FIGS. 12A-12D. Spacer microfibers 104 of ˜15 μm in diameter are introduced to strengthen the scaffold and separate the nanofiber 102 layers. The spacer fibers shown in these figures were introduced as a set of 3 layers adding up to ˜45 μm gap between the nanofiber layers on the collection frame 134, as shown in FIG. 4A and FIG. 13A. In this particular example, each spacer fiber layer consists of 3 microfibers at a distance of 11 mm in the X-Y plane, creating a boundary with 25 mm² area, followed by the second and third spacer fiber layers in orthogonal orientation to each other. In other embodiments, a single spacer fiber, such as a single 50 μm fiber could have also been used as spacer to achieve an overall 50 μm spacing between nanofiber layers. Nanofibers with an interfiber distance of ∧=100 μm were placed between the spacer fibers in the third layer, as shown in FIG. 4A and FIG. 13B (arrow 1). The combination of 3 spacer fiber layers and one nanofiber layer is defined as SET-1 (or) S1, as shown in FIG. 4A. Two sets are developed with nanofiber layers orthogonal to each other, as presented in FIG. 4B and FIGS. 13A-130. SEM (FIG. 4C) and optical microscope imaging (FIG. 14A-14B) confirmed a precise 100×100 μm² lattice. Further, using a rotatable collector frame (FIG. 1.2C(i) inset) as described herein, a controlled angle including but not limited to θ=30° and θ=60° between three nanofiber layers (FIGS. 4D-4F) is demonstrated.

One of the critical applications of well-organized fibrous scaffolds include 3D cell culturing and tissue engineering. In organisms, cells are exposed to a 3D ECM microenvironment, which includes soluble factors, fibrillar protein aggregates, and neighbor cells. They experience interactions with other cells and mechanical forces developed in dynamically changing organs. Outside this cell-specific microenvironment, cell functions could be alternated, as shown in numerous experiments. Obviously, biomimetic 3D ECM is the most efficient approach for tissue regeneration and the study of cell response to their microenvironment. GFD, in combination with additive manufacturing technology, offers a unique capability to mix fibers of different diameters, different materials (such as FDA approved polymers and biomaterials), and different mechanical properties at controlled spacing and orientation into the scaffold, thereby closely mimicking the natural ECM structure for cell differentiation, proliferation, and tissue development.

The ongoing research in the tissue engineering community relies on the ES approach to fabricate non-woven fibrous mats where the scaffold is filled with fibers leaving little volume for cell infiltration and 3D cell culture proliferation. The major problem of 3D ES is a poor control of the microstructure, specifically interfiber spacing. Porosity of nanofiber mats can be tuned in quite a broad range using sacrificial materials or pore-forming additives. However, this kind of porosity is different from the porosity of native tissues. A generated pore network in the nanofiber mats mimics microvasculature. However, in the pore walls, nanofibers remain densely packed and leave less space for cell infiltration and differentiation. Increased porosity provides an increase in the support for cell culture proliferation and guidance. However, a large fraction of the scaffold volume remains unavailable for cells in the pore walls amid the pore volume contains no supporting fibers. The effective number of fibers that provide mechanical cues and directional guidance remains low, as well as the overall porosity of such scaffolds.

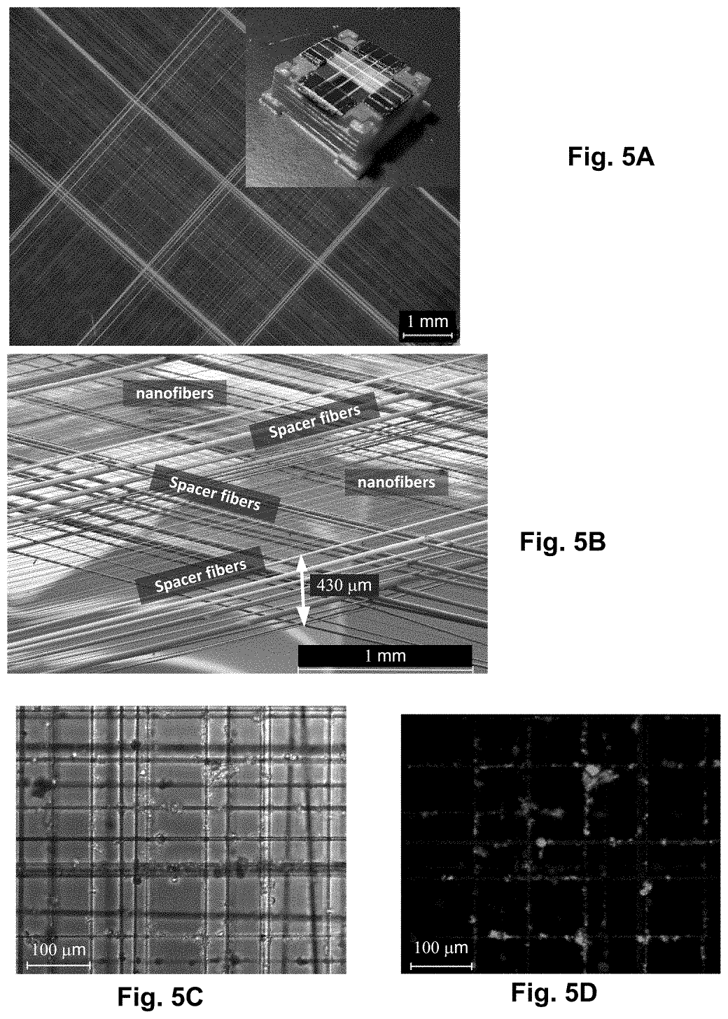

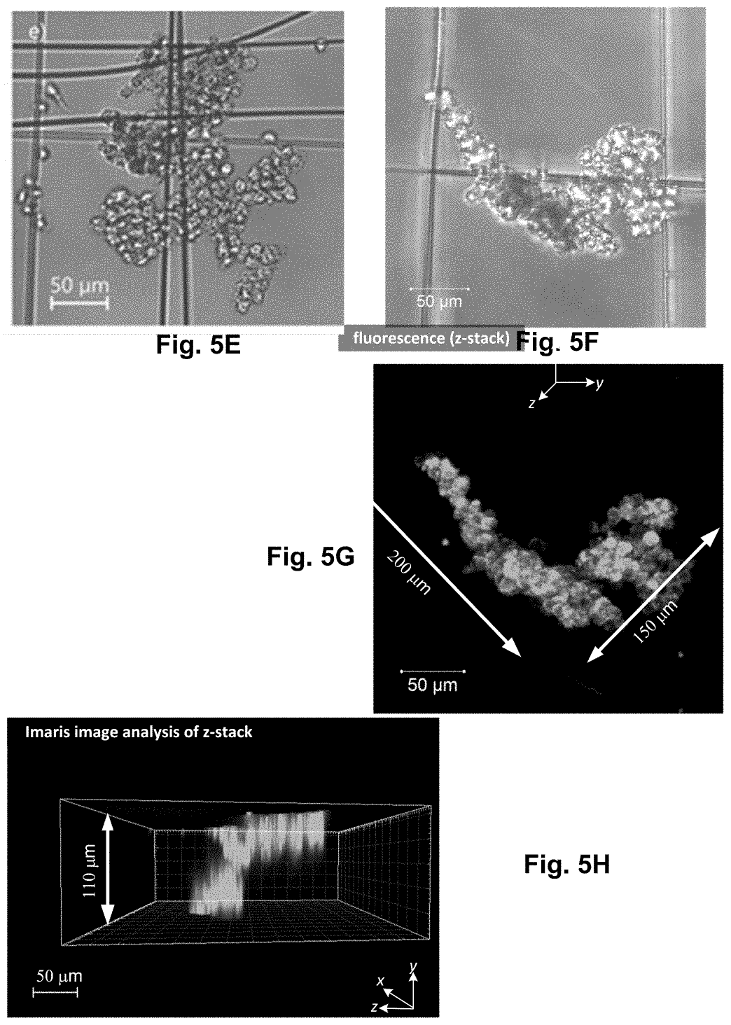

In general, cell infiltration through the nanofiber scaffold, anchoring on fibers, and proliferation are vital stages in a successful 3D cell culture application. To verify these basic features, a GFD PCL nanofiber scaffold was fabricated using the design scheme, as in FIG. 4A. The interfiber spacing and layer gap are 50 μm and 45 μm, respectively. This characteristic length is comparable with the mammalian cell size to secure cell infiltration and proliferation in the 3D scaffold structure. A scaffold thickness of 0.43 mm (FIGS. 5A and 5B) was achieved by the deposition of 8 sets of nanofiber layers.

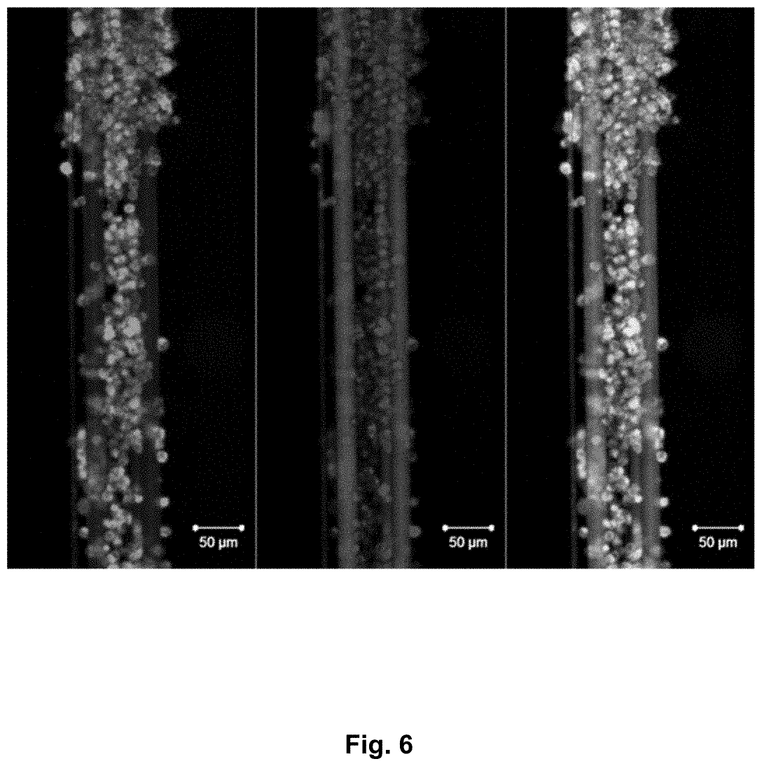

The porosity of the 3D scaffold presented in FIG. 3A is 99% when only 1% of the total 10.75 mm³ scaffold volume is occupied by the spacer and nanofibers. Hence, 99% of unoccupied space in the scaffolds is available for cell diffusion, nutrient and metabolite exchange, and cell growth. RAW 264.7 macrophages were cultured with this scaffold for 36 h. Then, the cells were stained with wheat germ agglutinin (WGA) and were imaged using a confocal microscope. Optical imaging (FIG. 5C) and the z-stack (depth 100 μm) confocal images (FIG. 5D) of the scaffold confirmed the cell diffusion, anchoring, and healthy proliferation (FIGS. 5E, 5F, and 5G) throughout the scaffold. The confocal image analysis software (Imaris) revealed the proliferation of macrophages in three-dimensional space (200×150×110 μm³) between the fiber layers of the scaffold (FIG. 5G). RAW macrophages infiltrate in the interfiber space and anchor to the fibers, as evident from FIG. 6 (dual FTIC channels). Macrophage engulfing is not observed as the cells are proliferated in a healthy state. The cell seeding experiments demonstrate the potential of GFD nanofiber scaffolds. A scaffold development plan mimicking structural features of ECM in different tissues is warranted for a successful tissue engineering application in the future.

In conclusion, described herein is a gravitational fiber drawing method to fabricate meter long freestanding monofilament nano- and microfibers of varying diameters (0.4 μm-100 μm) and a scaffolding device to handle the single filament fibers as building blocks to construct precisely organized 2D arrays and 3D scaffolds of the nanofibers using an additive manufacturing approach. The GFD method has the potential to develop precisely organized nanostructured materials by the bottom-up approach, offering flexibility for the use of different polymers and solvent systems, simple manufacturing processes, and scalability advantages. Using the methods described herein, it has been shown that meter long nanofibers could be held between anchors and manipulated by placing them in a linear or flat sheet-like structure. However, the technique is not limited to the linear scaffold structures. With a few modifications in the scaffolding instrument presented in this article, it is easy to wind or coil the fiber around a variety of geometries. For example, a tubular structure (closer to vascular graft) or a cube (closer to bone tissue) is easy to achieve with a different instrument design.

Other interesting features such as twisted, woven, and braided nanofiber fabrics are also feasible with the appropriate design of the scaffolding device. This work provides a solution for the fabrication of highly porous 3D scaffolds for cell cultures.

Experimental Section

PCL (Mw=80,000 g/mol) was purchased from Sigma. Chloroform and benzene (Sigma) were used as received. The GFD setup was fabricated in the lab (see Supporting information for details). The fiber dimensions and alignment was examined using optical and scanning electron microscopy.

RAW264.7 cells, a murine macrophage cell line, were used grown in RPMI-1640 medium with L-glutamine, containing 10% FBS and 0.99% penicillin-streptomycin. The cells were cultured in a humidity incubator at 37° C. with 5% CO2—commonly used parameters for tissue, cell culture experiments, and in-vitro assays. Previous experiments with PCL fibers demonstrated that PCL nanofibers undergo no changes within several day incubation time.^{[16]}

Before culturing cells, all polymeric scaffolds were pre-sterilized by immerged in 70% ethanol solution for longer than 3 h followed by PBS washes for three times. UV sterilization of scaffolds also proven successful as a dry alternative to 70% ethanol. Then, RAW264.7 cells were sub-cultured on top of the scaffolds, and the medium was added to submerge the scaffolds fully. Cells were cultured on the scaffolds for 5-6 days. Medium was changed every other day. At the end of cell culture, the medium component was removed, and the scaffolds were washed with cold PBS twice. Then the cells were fixed with formalin buffered saline for 5 min at room temperature and then washed with cold PBS twice. Finally, cells were stained with wheat germ agglutinin (WGA) for fluorescence imaging, and the FITC channel was used.

### Example 2

### Nanofiber 3D Scaffolding Device

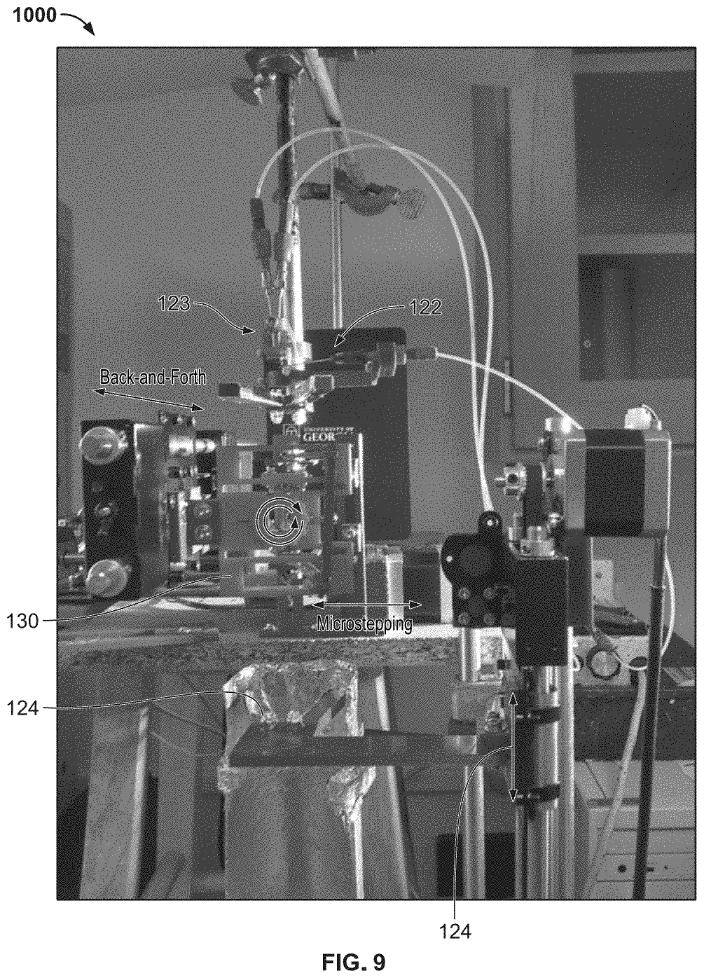

The scheme of the 3D scaffolding device 1000 prototype is presented in FIG. 7A-7B and FIGS. 8A-8D. The scheme of a nanofiber 3D array 100 (also referred to as a 3D nanofiber scaffold or 3DFS throughout) is presented in FIG. 7B. The in-lab developed device is shown in FIG. 9. The device 1000 is designed to have a fiber drawing assembly 110 (including the syringe 122 and downward moving tension stage 124), a fiber collection and delivery assembly 120 (including two poles or positioning arms 112), and a fiber positioning and scaffolding assembly 130 (including the fiber collector stage 132 and fiber collecting frame 134), as shown in FIG. 8A. In other embodiments, such as shown in FIG. 9, there may be more than one fiber drawing assembly 110. For example, one or more nozzles 122 can be included for drawing different types of fibers 102 or spacer fibers 104.

In the first part, the fiber collection and delivery assembly consists of fixed grooves 114 associated with the side-to-side flipping fiber positioning arms 116, fiber slicing blades 160, and needles 122 for drawing fibers 102 as pictured in FIG. 1A and FIGS. 8A-8D. In the second part, the tension stage 124 has a key function to hold the fiber in place with the help of anchors 126 and 128. A fixed anchor 126 is placed just below the needle with two arms that snap together upon activation with magnets (see detail of FIG. 15C in the dotted ring). The second anchor with two snapping arms is connected to an up and down movable stepper motor-controlled tension stage 124 and placed just above the base where droplets rest. The anchors 126 and 128 with the help of the tension stage 124 are helpful in straightening the nanofiber 102 and holding it for collection. The fixed positioning assembly 112 and tension stage 124 work in sequence to position the nanofiber 102 in the fixed space. The positioned fiber 102 is then collected by the moving stage 132 which acts as a precision traveling fiber collector to collect fibers 102 on its collection frame 134. The fiber positioning and scaffolding assembly 130 has a slot to accommodate the replaceable nanofiber collection frame 134, a screw or rotational handle 136, and gear mechanism to rotate the collection frame 134 to any desired angle, fiber cutting trenches above and below the collection frame slot to allow the fixed stage blades 160 to pass through. The precise motion of the fiber positioning and scaffolding assembly 130 is achieved via two stepper motor controlled linear travel stages, each in x-direction and y-direction. The fiber positioning and scaffolding assembly 130 is directly connected to the x-travel stage for a rapid and smooth back-and-forth motion in the y-direction. This y-travel stage is fixed on another precision microstepping linear travel stage in the y-direction. The assembled working device is shown in FIG. 9.

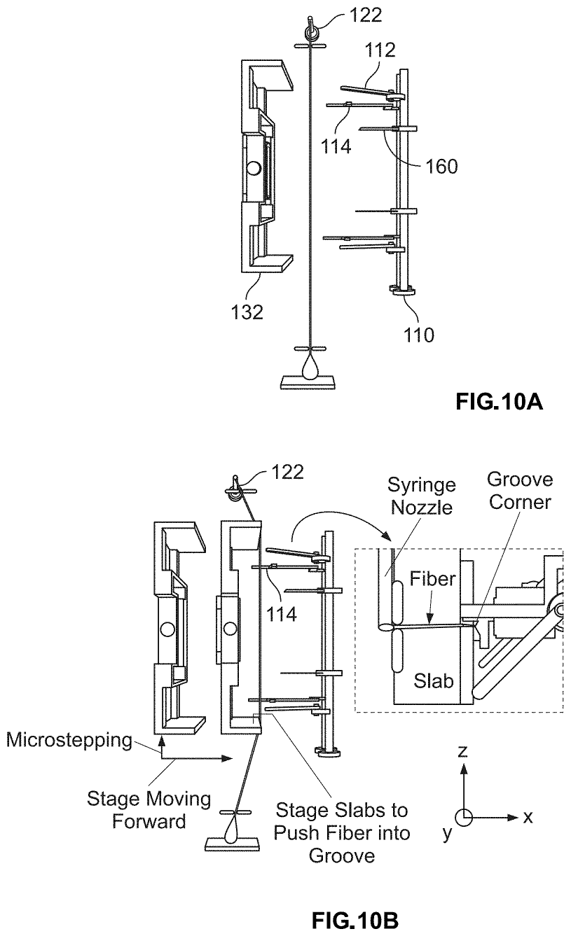

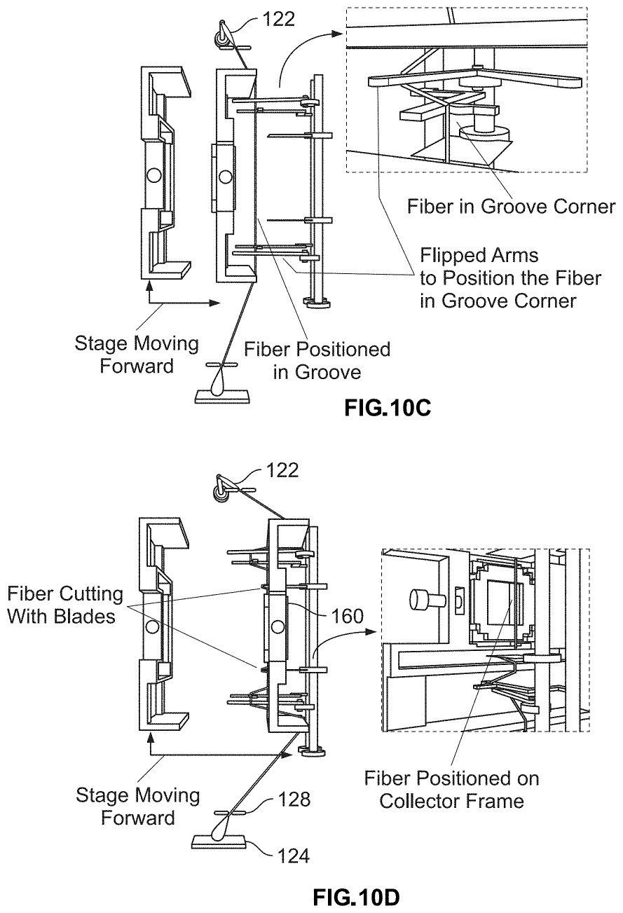

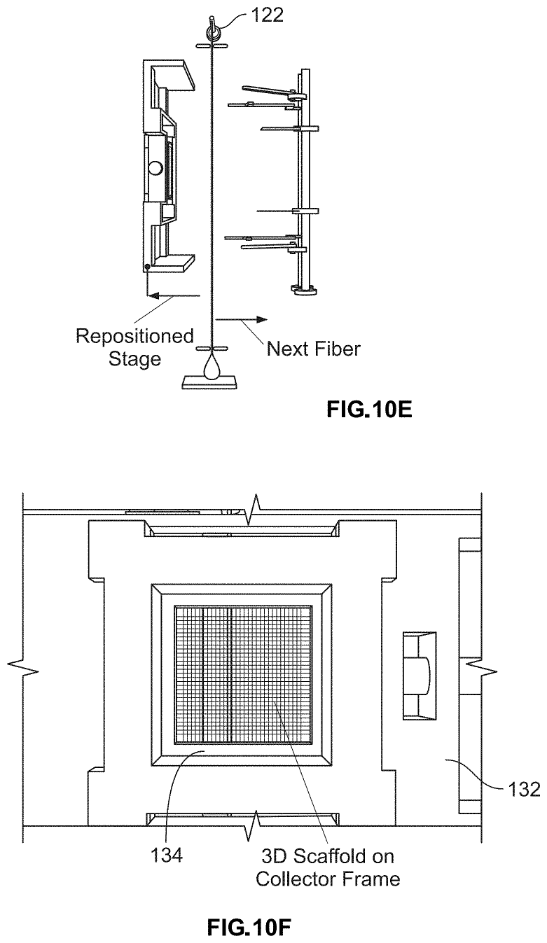

FIGS. 10A-10F provides an overview of the fiber scaffolding process using a device 1000 as described in FIGS. 8A-8D or FIG. 9. FIG. 10A shows a front view of the device 1000 at the first stage of fiber drawing, in which a droplet has been dropped from the nozzle 122 onto base 124 to draw a fiber 102. In FIG. 10B, the slabs on moving fiber collector stage 132 have pushed fiber 102 into grooves 114 on fiber positioning arms 112. In FIG. 100, flip arms 116 further secure the fiber 102 in place as stage 132 moves further forward. In FIG. 10D, the stage 132 has moved forward so that the fiber 102 is positioned on collector frame 134 and blades 160 cut the fiber. With the fiber 102 adhered to the collector frame 134 (FIG. 10E), the stage 132 can return to its original position, microstep laterally, and the process can begin again to draw, cut, and collect further fibers. FIG. 10F shows a 3D scaffold 100 formed from numerous fibers 102 collected on frame collector frame 134 on stage 132.

Cold Drawing of Single Filament Fiber

In this experiment, a single filament fiber from a single falling droplet is used to demonstrate the capability of the GFD technique to achieve different fiber draw ratios (FIG. 15A-15F). A linear motion rail (FIG. 15C) with two anchors each at bottom and top is used to perform the fiber drawing process. The bottom anchor (in a dotted ellipse, FIG. 15C) is fixed, and the top anchor is movable in up and down directions. Each anchor is mounted with two magnetic snap arms to hold the fiber in place between the anchors. A single droplet of 10% PCL solution is allowed to fall from the syringe needle, fabricating a 20 cm long initial fiber (FIG. 15A). The fiber is held between the anchors, followed by the collection of a fiber section for measuring the diameter. The remaining fiber is drawn continuously in different stages as demonstrated in FIGS. 15A and 15C. The diameter of the corresponding fibers from each draw stage is measured using SEM images. Draw ratios are estimated based on the initial and final fiber diameter at each stage. A 20 cm long PCL fiber with 1.6 μm initial diameter is divided into sections and stretched to a draw ratio of 10.24 before it reaches a breaking point.

### Example 3

### Spiral Winding of Nanofibers

To extend the 3D scaffolding capabilities such as productivity, reproducibility, scalability, and applications of the device described in Example 2, and to overcome the limitations of scaffold geometries, described herein is a spiral winding approach utilizing the same gravitational fiber drawing (GFD) technique. In this approach, meter-long gravity drawn nanofibers are coiled around a supporting frame of virtually unlimited geometries (e.g. tube, cylinder, square, cube, etc.). This technique can be used to create a scaffold for example that mimics an extracellular microenvironment (ECM) and vascular channels of living tissues for in vitro 3D cell cultures in cancer and stem cell research, tissue engineering and regenerative medicine. Other applications of these scaffolds can include nanoparticulate filters, smart textiles, sensors, microelectronics etc. The multistep process can be automated or semi-automated using devices described herein.

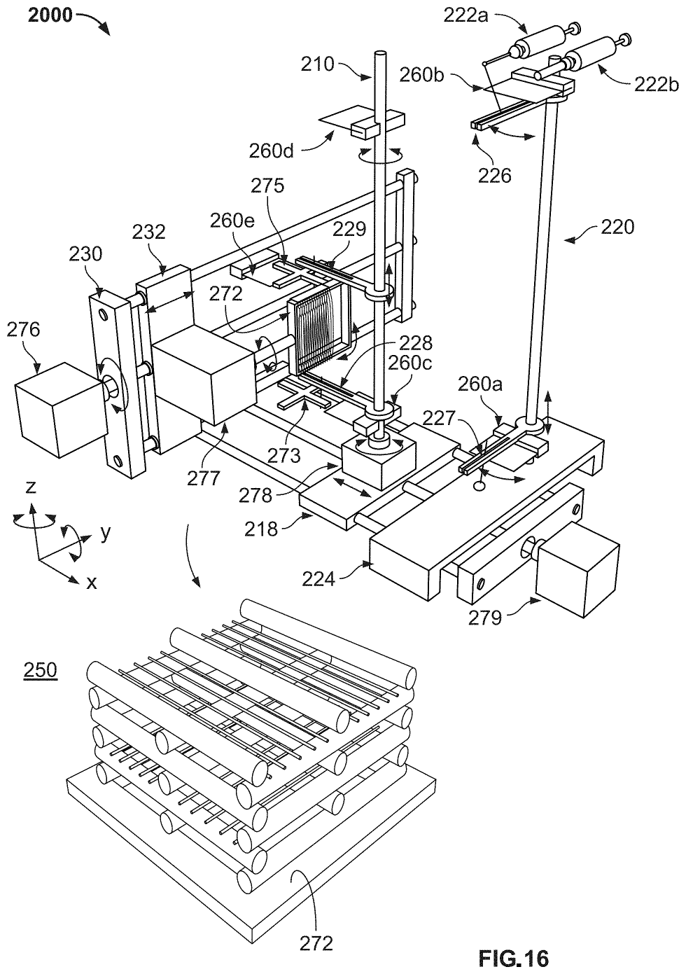

An example scheme of a spiral-winding device is presented in FIG. 16. Some processing stages associated with this device 2000 include drawing a fiber, anchoring the drawn fiber, delivering the fiber for spinning/winding/coiling around a frame/post, and repeating these three processes until the desired 3D scaffold 250 is achieved. The device design was developed to achieve the sequence of these processes. The device 2000 contains a fiber drawing assembly 220 including a fiber collection and delivery assembly 210 with anchors at top and bottom, and a fiber positioning and scaffolding assembly 230 that includes a microstepping linear motion stage 232 with sample/frame holder 272 for 3D scaffolding 250.

### Fiber Drawing Assemblies:

Turning now to FIG. 16, an embodiment of the fiber drawing assembly 220 is described. In this example, the fiber collection and delivery assembly 210 is integrated with the fiber drawing assembly 220. The fiber drawing assembly 220 is equipped with polymer solution droplet extruding needles/nozzles 223 (e.g. syringe needle), two or more fiber anchors 226 and 227, and fiber cutting blades 260a-e (the blades can be other types of fiber cutter, e.g. hot blade, hot wire, etc.) connected to a vertical rod or similar structure fixed on a base 224. Each fiber anchor can have two arms, arm 212 is fixed in position and arm 216 can have a movable/flip motion. These arms can snap together upon actuation to hold the fiber 202 in place (e.g. magnet based mechanical snap, etc.). The anchor 227 at the bottom of the rod is designed to move up and down with the help of a z-axis linear motion stage or similar motion controlling device. The fiber anchor 226 at the top of the rod is fixed to remain in position. However, either anchor 227 or 226 could be chosen to be movable or fixed or in combination to hold and straighten the fiber drawn from the falling droplet. Both anchors are aligned/positioned in parallel in the same orientation. A cartridge of polymer solution extruding nozzles (222a, 222b etc) can be placed at the top of the rod and used to draw fibers with different materials, mechanical properties, and diameters. Polymer solutions can be fed to the needles/nozzles through a syringe pump or similar mechanism. The desired polymer solution can be pumped through the nozzle 223 until a single droplet is extruded and prepared to leave the nozzle tip. The downward traveling droplet leaves behind a trail of hanging fiber as it reaches the base 224 (see also Examples 1 and 2 above). Instantly after the droplet reaches the base, the anchors can actuate to snap their arms and hold the hanging nanofiber 202. Following the fiber anchoring, associated blades 260a, 260b can be activated to slice the fiber 202 below anchor 227 and above the anchor 226.

### Fiber Collection and Delivery Assembly

The fiber drawn and held in the previous process/stage between the anchors 227 and 226 can now be collected and delivered by a back-and-forth movable and rotatable vertical rod mechanism (fiber collection and delivery assembly 210) mounted on a stepper 278, together fitted onto a x-axis linear motion stage 218, attached with two or more fiber anchors 228 and 229 and fiber cutting blades (260 c, d; e.g. hot blade, hot wire, etc.). The first anchor 228 positioned at the bottom of the rod can be fixed in place. The second anchor 229 positioned at the top of the rod can be movable in up and down motion with the help of z-axis linear motion stage or similar mechanism. Both the anchors can be positioned in parallel orientation as shown in fiber collection and delivery assembly 210. The stepper 278 can be positioned on an x-axis linear motion stage which allows the movement of connected rod system along with stepper 278 to move back-and-forth towards the fiber drawing stage 224 to collect the fiber 202 and backwards to the fiber positioning and scaffolding assembly 230 to deliver the fiber on demand.

In the fiber collection and delivery assembly 210, the anchors 228 and 229 can be oriented/pointed towards the fiber drawing stage 224 with their snap arms 216 in open position. The fiber collection and delivery assembly 210 is then moved on x-axis linear motion stage 218 to reach the hanging fiber between anchors 226 and 227 (prepared during the fiber drawing). As soon as the anchors 228 and 229 reach the hanging fiber 202, the arms are snapped to hold the fiber in preparation for the collection process. Followed by the fiber anchoring between 228 and 229, the blades c3 and c4 will be activated to section/cut/slice the fiber above anchor 229 and below anchor 228. The fiber is now collected/separated from anchors 227 and 226 in the fiber drawing stage and held between 228 and 229 in the collection and delivery stage. In the next step, the fiber 202 is turned with a stepper motor towards fiber positioning and scaffolding stage. The fiber delivery system is then moved on from the x-axis linear motion 218 portion of the process towards the fiber positioning and scaffolding assembly 230 to deliver the fiber 202 for scaffolding.

In alternative embodiments, different configurations are possible, including fiber anchors 227 and 226 placed for collection at a height which eliminates the need for multiple rounds of cutting. In some embodiments, blades 206a and 206b can be used while the fiber is being delivered to the spiral winding stage, and another fiber can be developed simultaneously at the fiber drawing stage.

### Fiber Positioning and Scaffolding Assembly

The fiber positioning and scaffolding assembly 230 allows the fiber scaffolding process to be performed around a supporting frame 272 of any 2D and/or 3D geometry (e.g. square, tube, cylinder, cube, etc). This can be achieved by connecting the supporting frame 272 to a stepper motor 277 to achieve rotating motion around the y-axis. The rotating stepper 277 is then connected to a y-axis linear motion stage 232. In this design, the fiber collection and delivery assembly 210 with a hanging fiber 202 collected by anchors 228 and 229 is moved towards the supporting frame 234. Sacrificial adhesive material can be used at one corner of the supporting frame to prepare contact of nanofiber and frame 234, followed by the fiber winding process.

The fiber winding around the frame with precision fiber spacing and alignments can be achieved by synchronizing the precision ‘microstepping’ of y-axis linear motion stage with stepper 276, rotational motion of frame 234 with stepper 277, z-axis motion of anchor 229 with stepper 279 while the fiber is being coiled around the frame. In addition, other components to position the fiber in space are the grooves in support arms 273 and 275 and blades 260c,e around the supporting frame. More details of the groove design and functionality is described Example 2 (FIGS. 8A-8D).

The processes and device operation described in the present example can be repeated as many times needed until the entire 3D scaffold is fabricated as desired. Post scaffolding, the edges/ends of 3D scaffolds can be secured permanently on the supporting frame with biocompatible adhesives or thermal processes. As an alternative, the 3D scaffold can be collected by external frames/inserts/cups suitable for using with multi-well plate or a T-flask, or as cartridge for perfusion bioreactor system. These scaffolds with suitable fiber materials are highly desired such as for 3D cell culture applications to be used in suitable culture flasks.

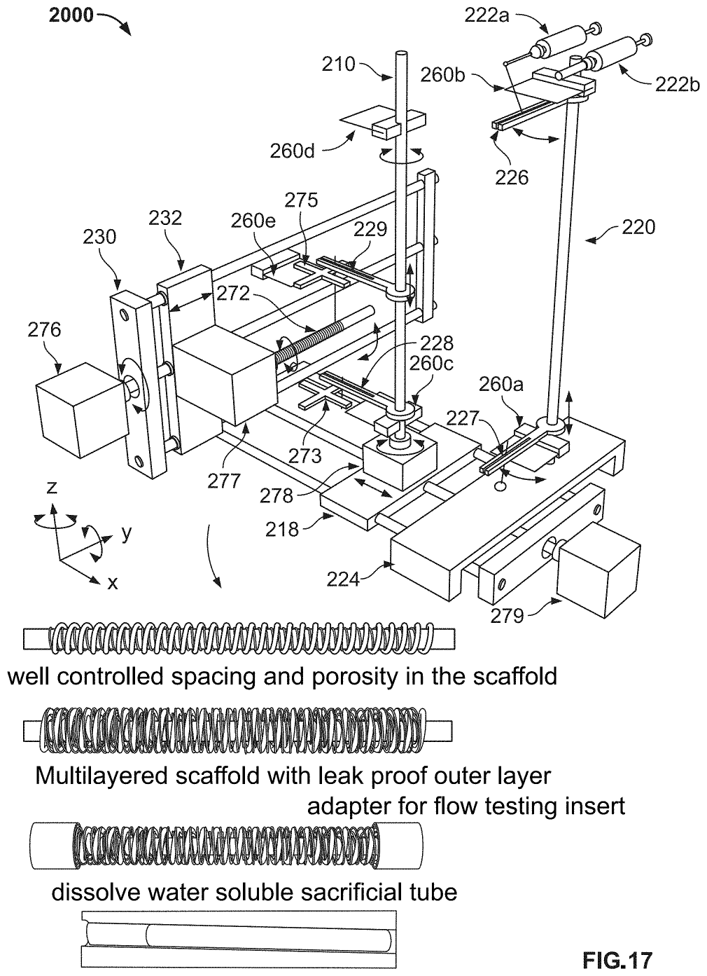

Turning now to FIG. 17, in an alternative approach, a water-soluble sacrificial frame (such as including polyvinyl alcohol) can be used to form a tubular scaffold instead of permanently fixing the scaffold on supporting frame as previously described. Such an approach can be used for tissue engineering applications such as vascular grafts, amongst others. The main difference between the devices presented in FIGS. 16 and 17 is the supporting frame attached to the stepper 277; in FIG. 17 the scaffold is a tubular rod, rather than a frame. Post scaffolding, the tubular frame can be connected to adapters at the ends followed by dissolving the frame with running water/buffer to obtain the fiber-based 3D scaffold in the desired geometry. In some embodiments, the resulting scaffold can then be ready for cell diffusion and tissue development in a bioreactor.

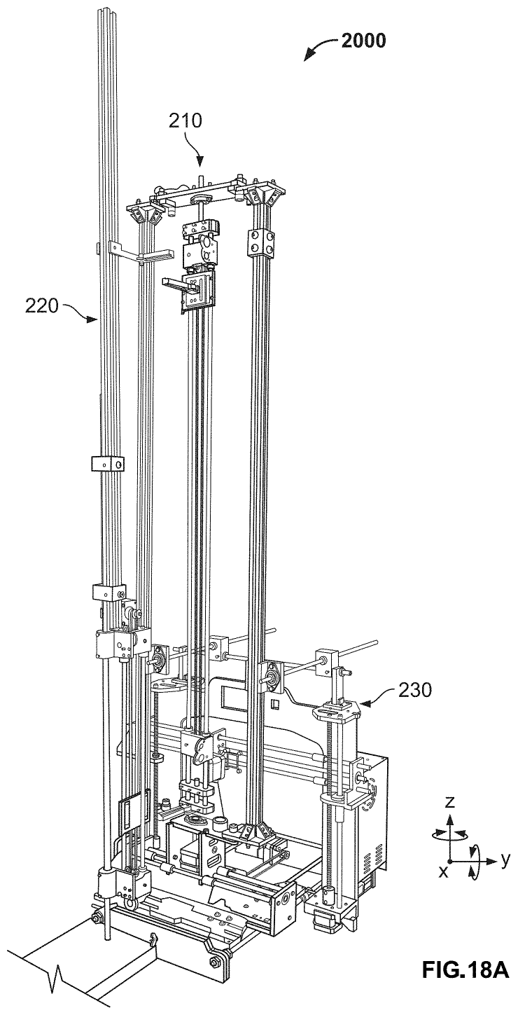

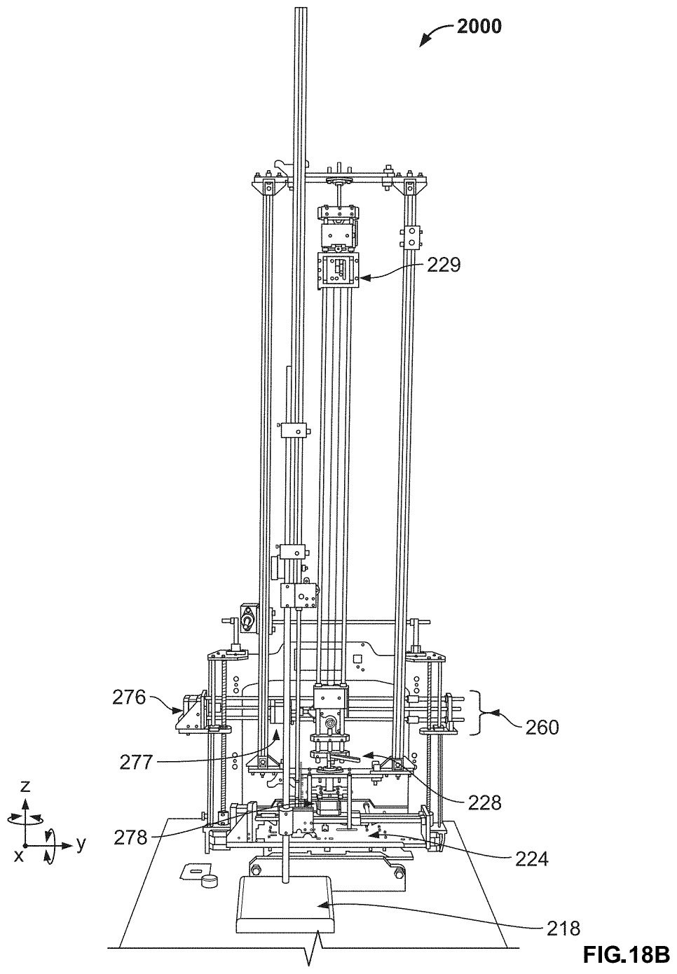

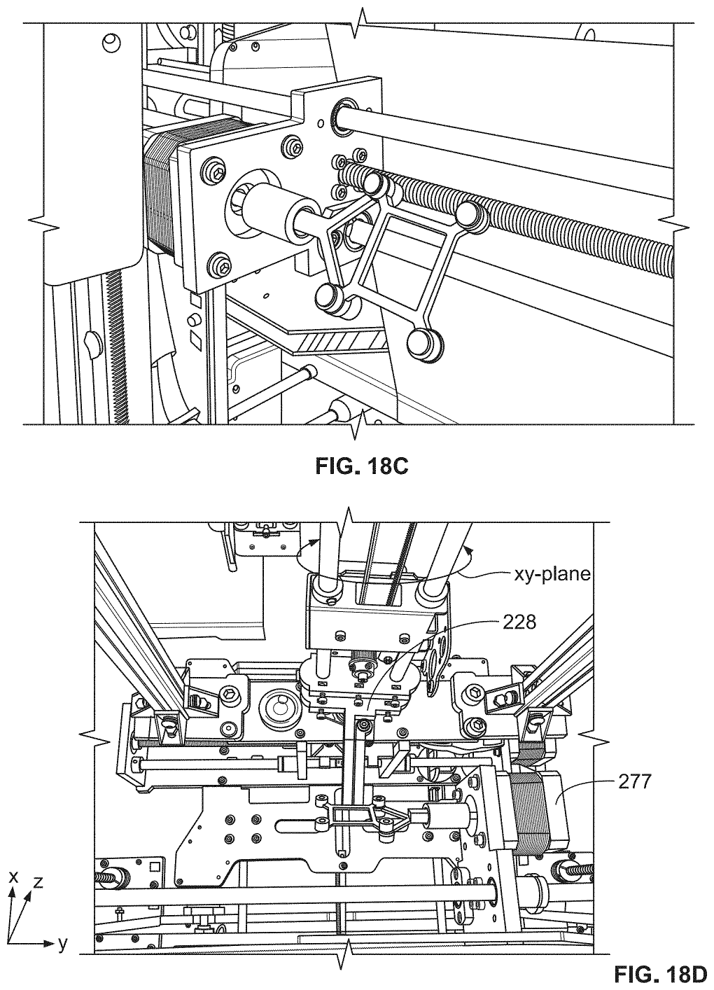

A prototype version of device designs presented in FIGS. 16 and 17 is shown in FIGS. 18A-18D. As can be envisioned by one of skill in the art, various configurations could be used based on the processes and assemblies described above.

### Example 4

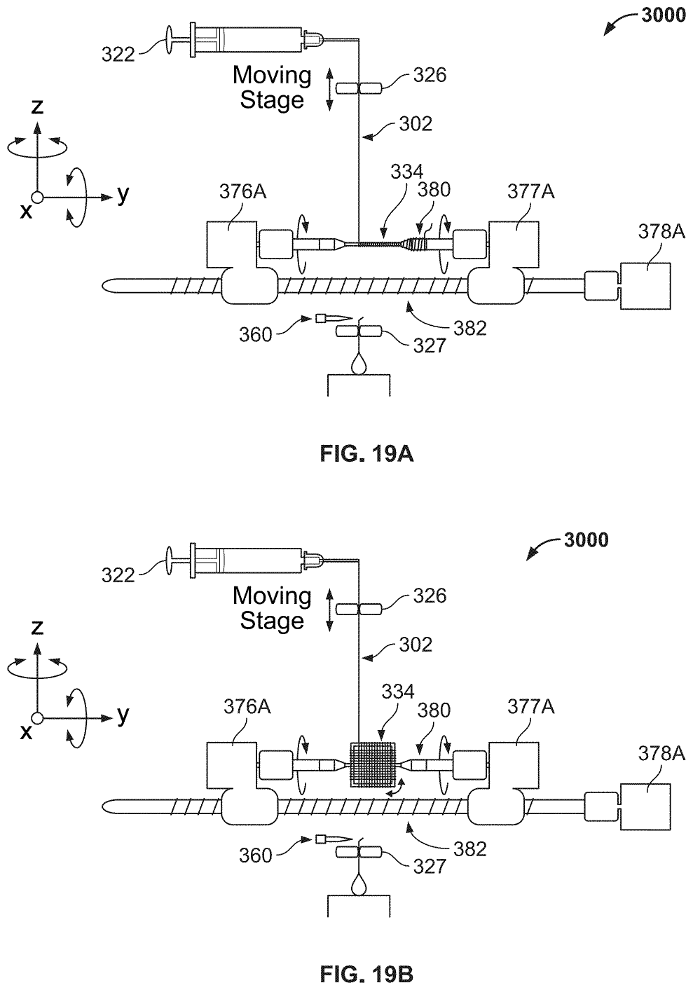

When scaffolds of large dimensions (>10×10 cm²) are desired, a device design such as the one presented in FIGS. 19A-19B could be used. This device 3000 follows the same processing stages presented in Example 3. The droplet is ejected by nozzle 322 to form fiber 302 and held in place by anchors 326 and 327. However, the fiber collector frame 334 can be equipped/held with two steppers 376, 377 instead of a single motor for turning fiber collector frame 334. Microstepping screw 382 can move the fiber collector frame 334 linearly. Adhesive 380 can adhere fiber collector frame 334 temporarily to the device. Such a device could be used to improve the stabilities and precision of fiber scaffolding for use in industrial scale 3D cell culture or other applications such as nanoparticulate filters.

### Example 5

### Commercial Scale Production of Scaffolds

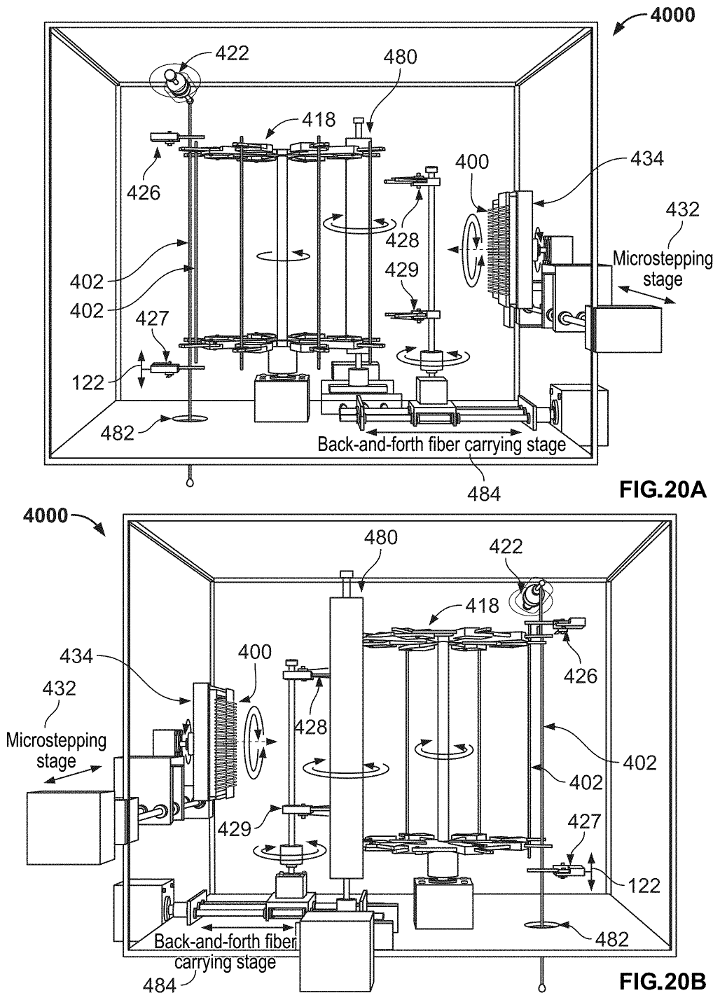

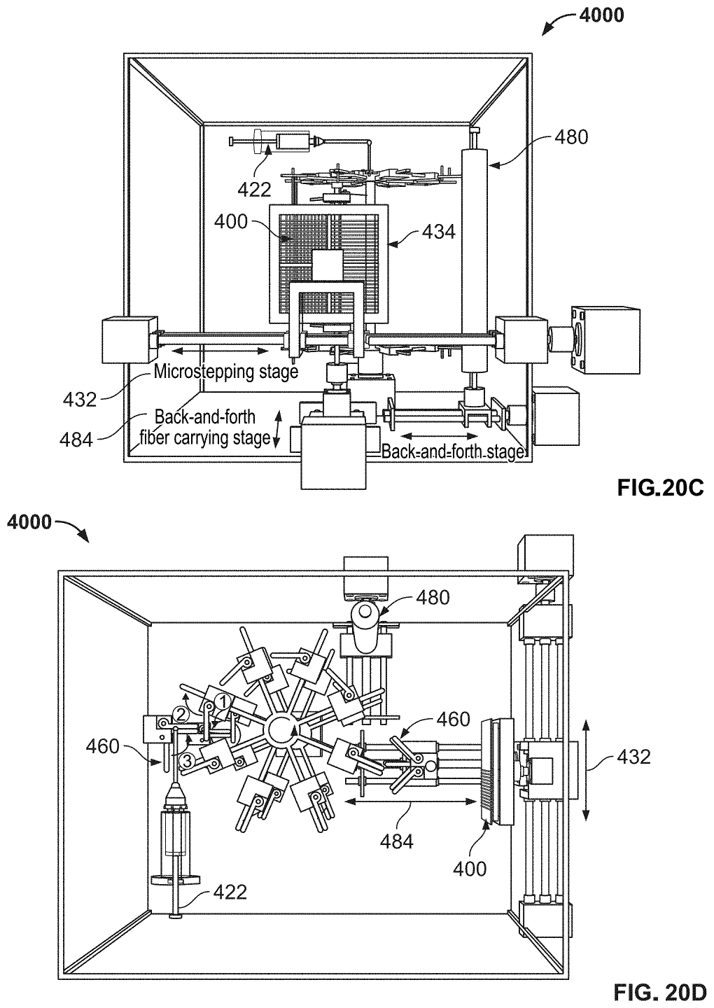

In addition to the instrument design, another major hurdle for rapid fabrication of 3D scaffolds at commercial scale is the fiber drawing process. Described herein is a continuous fiber production/drawing and storage system as presented in FIGS. 20A-20D The fibers drawn from the falling droplets can be collected by a storage and delivery wheel 418 (FIGS. 20A-20D). The fibers are collected on arms attached to the wheel having fiber anchors and blades. As the wheel rotates, one fiber is collected as another is delivered to a fiber collection and delivery assembly for cutting and transfer to a 3D scaffolding assembly. In some embodiments, each fiber can be drawn to a specific length, such as one meter. In some embodiments, more than one fiber storage and delivery wheel 418 can be included, allowing preparation of fibers of different lengths (such as about 20 cm to about 200 cm). Combining this storage wheel with a fiber drawing assembly including a cartridge having polymer solution-extruding nozzles 422 is a powerful tool to create highly customized and precision aligned 3D scaffolds. The combination allows for the capability to produce fibers of different diameters, mechanical properties, materials, and the ability to deliver them in sequence to form a 3D scaffold that can be used for applications including 3D cell culture and tissue engineering applications.

The fiber collection and delivery assembly (including storage and delivery wheel 418, and fiber anchors 428 and 429) as presented in Example 3 can go rapidly back-and-forth to collect the fibers from the storage wheel and deliver them to the 3D scaffolding assembly (supporting frame). Any of the embodiments described in the examples herein could be combined with the one or more fiber storage wheel assemblies for rapid 3D scaffolding. FIGS. 20A-D specifically show a version of the device described in Example 2 coupled with the storage wheel. FIGS. 20A-D also show a stray fiber dusting cylinder 480 that can be used with the storage wheel. This cylinder can blow air/spin to direct air flow/other to remove loose cut ends of fibers from the device to prevent them becoming incorporated into the fiber positioning and scaffolding assembly 400, which can include microstepping stage 432 and fiber collector frame 434. The elements in various embodiments described herein, such as the storage wheel, can be programmed to operate as demanded by the scaffold design.

### REFERENCES

[1] Z. Wang, S. M. Mithieux, A. S. Weiss, Adv Healthc Mater 2019, 8, e1900742.
[2] a) A. L. Yarin, B. Pourdeyhimi, S. Ramakrishna, Fundamentals and Applications of Micro and Nanofibers, Cambridge University Press, Cambridge 2014; b) V. Beachley, E. Katsanevakis, N. Zhang, X. Wen, in Biomedical Applications of Polymeric Nanofibers, DOI: 10.1007/12_2011_141 (Eds: R. Jayakumar, S. Nair), Springer Berlin Heidelberg, Berlin, Heidelberg 2011, Ch. Chapter 141, p. 171; c) J. H. Park, G. C. Rutledge, Macromolecules 2017, 50, 5627.
[3] a) X. Li, X. X. Wang, T. T. Yue, Y. Xu, M. L. Zhao, M. Yu, S. Ramakrishna, Y. Z. Long, Polymers 2019, 11; b) M. Cao, F. Gu, C. Rao, J. Fu, P. Zhao, Sci. Total Environ. 2019, 666, 1011; c) A. Rajak, D. A. Hapidin, F. Iskandar, M. M. Munir, K. Khairurrijal, Nanotechnology 2019, 30, 425602; d) H. Liu, J. Huang, J. Mao, Z. Chen, G. Chen, Y. Lai, iScience 2019, 19, 214.
[4] M. Gorji, R. Bagherzadeh, H. Fashandi, in Electrospun Nanofibers, DOI: 10.1016/b978-0-8-100907-9.00021-0 (Ed: M. Afshari), Woodhead Publishing 2017, p. 571.
[5] a) K. Song, Q. Wu, Y. Qi, T. Kärki, in Electrospun Nanofibers, DOI: 10.1016/b978-0-8-100907-9.00020-9 (Ed: M. Afshari), Woodhead Publishing 2017, p. 551; b) D. Simoes, S. P. Miguel, M. P. Ribeiro, P. Coutinho, A. G. Mendonca, I. J. Correia, Eur. J. Pharm. Biopharm. 2018, 127, 130.
[6] a) J. H. Kim, J. Y. Park, S. Jin, S. Yoon, J. Y. Kwak, Y. H. Jeong, Nanomaterials 2019, 9, 588; b) L. Jin, Q. Xu, S. Kuddannaya, C. Li, Y. Zhang, Z. Wang, ACS Appl. Mater. Interfaces 2017, 9, 17718.
[7] a) R. Rasouli, A. Barhoum, M. Bechelany, A. Dufresne, Macromol. Biosci. 2019, 19, e1800256; b) X. Zhang, C. Wang, M. Liao, L. Dai, Y. Tang, H. Zhang, P. Coates, F. Sefat, L. Zheng, J. Song, Z. Zheng, D. Zhao, M. Yang, W. Zhang, P. Ji, Carbohydr. Polym. 2019, 213, 27; c) S. Aslani, M. Kabiri, M. Kehtari, H. Hanaee-Ahvaz, J. Cell. Physiol. 2019, DOI: 10.1002/jcp.28266; d) Z. Arabpour, A. Baradaran-Rafii, N. L. Bakhshaiesh, J. Ai, S. Ebrahimi-Barough, H. Esmaeili Malekabadi, N. Nazeri, A. Vaez, M. Salehi, F. Sefat, S. N. Ostad, J. Biomed. Mater. Res. A 2019, DOI: 10.1002/jbm.a.36742; e) N. Saveh-Shemshaki, S. N. L, C. T. Laurencin, Acta Biomater 2019, 94, 64.
[8] a) S. T. Aruna, L. S. Balaji, S. S. Kumar, B. S. Prakash, Renewable and Sustainable Energy Reviews 2017, 67, 673; b) Z. Liang, G. Zheng, C. Liu, N. Liu, W. Li, K. Yan, H. Yao, P. C. Hsu, S. Chu, Y. Cui, Nano Lett. 2015, 15, 2910; c) L. Xia, S. Wang, G. Liu, L. Ding, D. Li, H. Wang, S. Qiao, Small 2016, 12, 853.
[9] a) D. Ye, Y. Ding, Y. Duan, J. Su, Z. Yin, Y. A. Huang, Small 2018, 14, e1703521; b) X. Wang, F. Sun, G. Yin, Y. Wang, B. Liu, M. Dong, Sensors 2018, 18; c) W. Jayathilaka, K. Qi, Y. Qin, A. Chinnappan, W. Serrano-Garcia, C. Baskar, H. Wang, J. He, S. Cui, S. W. Thomas, S. Ramakrishna, Adv. Mater. 2019, 31, e1805921.
[10] M. Zhu, Y. Wang, S. Zhu, L. Xu, C. Jia, J. Dai, J. Song, Y. Yao, Y. Wang, Y. Li, D. Henderson, W. Luo, H. Li, M. L. Minus, T. Li, L. Hu, Adv. Mater. 2017, 29.
[11] a) D. Shou, J. Fan, Langmuir 2018, 34, 1235; b) S. M. Park, Y. S. Huh, K. Szeto, D. J. Joe, J. Kameoka, G. W. Coates, J. B. Edel, D. Erickson, H. G. Craighead, Small 2010, 6, 2420.
[12] a) H. Lee, D. Kharaghani, I. S. Kim, in Novel Aspects of Nanofibers, DOI: 10.5772/intechopen.73521 2018, Ch. Chapter 1; b) K. Watanabe, B.-S. Kim, Y. Enomoto, I.-S. Kim, Macromol. Mater. Eng. 2011, 296, 568; c) A. S. Nain, M. Sitti, A. Jacobson, T. Kowalewski, C. Amon, Macromol. Rapid. Commun. 2009, 30, 1406; d) A. S. Nain, J. C. Wong, C. Amon, S.-M. Letters, Appl. Phys. Led. 2006; e) X. Bai, S. Liao, Y. Huang, J. Song, Z. Liu, M. Fang, C. Xu, Y. Cui, H. Wu, Nano Lett. 2017, 17, 1883; f) A. Tokarev, O. Trotsenko, I. M. Griffiths, H. A. Stone, S. Minko, Adv. Mater. 2015, 27, 3560; g) A. Tokarev, D. Asheghali, I. M. Griffiths, O. Trotsenko, A. Gruzd, X. Lin, H. A. Stone, S. Minko, Adv. Mater. 2015, 27, 6526.
[13] a) J. A. Matthews, G. E. Wnek, D. G. Simpson, G. L. Bowlin, Biomacromolecules 2002, 3, 232; b) J. I. Kim, T. I. Hwang, L. E. Aguilar, C. H. Park, C. S. Kim, Sci. Rep. 2016, 6, 23761; c) H. Pan, L. Li, L. Hu, X. Cui, Polymer 2006, 47, 4901.
[14] T. D. Brown, P. D. Dalton, D. W. Hutmacher, Adv. Mater. 2011, 23, 5651.
[15] a) G. Zheng, J. Jiang, D. Wu, D. Sun, in Electrospinning: Nanofabrication and Applications, DOI: 10.1016/b978-0-323-51270-1.00009-1 (Eds: B. Ding, X. Wang, J. Yu), William Andrew Publishing 2019, p. 283; b) X.-X. He, J. Zheng, G.-F. Yu, M.-H. You, M. Yu, X. Ning, Y.-Z. Long, J. Phys. Chem. C 2017, 121, 8663.
[16] a) D. Asheghali, S. J. Lee, A. Furchner, A. Gruzd, S. Larson, A. Tokarev, S. Stake, X. Zhou, K. Hinrichs, L. G. Zhang, S. Minko, Nanomedicine 2020, DOI: 10.1016/j.nano.2020.102152102152; b) S. J. Lee, D. Asheghali, B. Blevins, R. Timsina, T. Esworthy, X. Zhou, H. Cui, S. Y. Hann, X. Qiu, A. Tokarev, S. Minko, L. G. Zhang, ACS Appl Mater Interfaces 2020, 12, 2067.

### Example 6

### Fabrication and Assembling of a Prototype Fiber Fabrication and Scaffolding Device for 3D Fiber Scaffold (3DFS) Manufacturing.

The methods and devices described in the examples above may be used to manufacture 3D fiber scaffolds (3DFS) for cell culture and proliferation. Cell therapies (CT) promise to cure diseases and have the potential to innovate and improve the health care industry to benefit patients who suffer from long-lasting organ failure or dysfunctions, including complications caused by devastating epidemic cases such as the recent COVID-19 pandemic. The promising results of the studies of Chimeric antigen receptor T-cell (CAR-T)-based autologous cell therapies in curing cancers permanently¹ and efficient cure for other diseases demonstrates the need for enabling the CT industry through better cell manufacturing tools. This ensures the rapid and timely commercialization of effective cellular therapeutic products for many chronic diseases like cardiovascular, liver, kidney, and neurodegenerative, pancreatic dysfunctions, etc. The scaling-up of the manufacturing of healthy adherent cells and their harvesting with minimal or no damage and contamination has been a bottleneck issue for the CT industry since the beginning.

Described here is a fabrication method of highly porous 3D fiber scaffolds made from precision aligned and stacked nanofiber arrays. These scaffolds, which can support cell infiltration, can be customized with spatially organized cell-specific physical and biochemical cues, microporous structure for flow of media, and secure, simple harvesting of cells with mechanical cell strainers. These 3DFS can be used for a single-use, plug-and-play style operation with a dynamic culture system in a modular perfusion bioreactor.

The major limitation for the application of well-defined 3D fiber scaffolds has been caused by the absence of an appropriate nanofabrication method. Electrospinning (ES), near-field electrospinning (NFES), molding and 3D-printing are not capable of providing 3D filament structures that combine well-controlled fiber diameter, interfiber spacing, their alignment, organization, biochemical cues, and mechanical characteristics in 3D-directions across the length scale that match the morphology of native tissues.²⁻⁷ These known methods provide scaffolds with various limitations. The limitations include, low porosity and very small interfiber spacing for cell infiltration (by ES methods), a combination of domains of high porosity with a very large interfiber spacing and domains with a very low interfiber spacing (by methods of ES with sacrificial pore-forming materials), or well-aligned but very thin (a few mm thick) scaffolds (by NFES methods), or well-aligned scaffolds with a larger fiber diameter (by 3D-printing methods). ES, NFES, and 3D-printing techniques are mainly limited to some tissue engineering applications and less suitable for scale-up manufacturing of adherent cell culture. The methods and devices here may solve these problems.

Nanofibers are primary structural components of the extracellular matrix (ECM), providing physical and biochemical cues for the healthy proliferation of most adherent cell types. The methods and devices herein allow for the fabrication of highly porous ECM-like 3D fiber scaffolds (3DFS) made of precision aligned and stacked nanofiber arrays. Commercially available electrospinning techniques are not suitable for the fabrication of large 3DFS geometries (e.g., 15×15×15 cm³) with high precision fiber spacing, 3D alignment, and >90% scaffold porosity. The gravitational fiber drawing (GFD) approach combined with herein disclosed scaffolding devices address these challenges. GFD-based scaffolding is an efficient nanofiber-drawing technology that allows for the reproducible fabrication of well-defined large nanofibrous 3D-structures. The 3DFS fabrication technology disclosed here is capable of generating fibrous geometries having a volume of over 5000 cm³ per scaffold while retaining over 90% porosity and providing >2 m² of cell attachment surface to accommodate over 4 billion cells per scaffold.

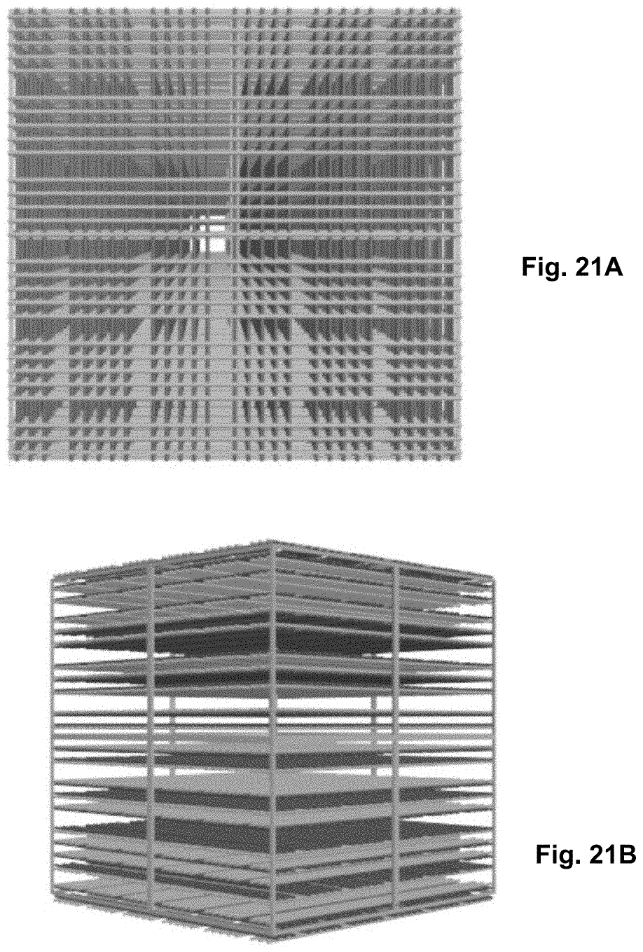

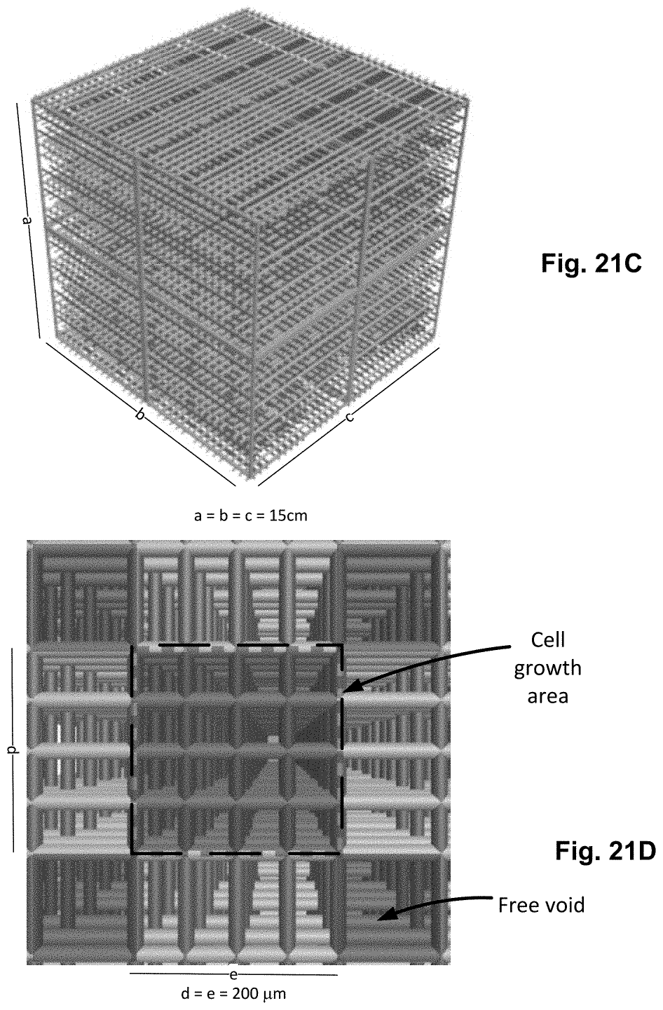

As described herein, 3DFS scaffolds can be formed that provide stacked nanofiber arrays having defined spacing between layers and a cage for controlled cell proliferation and free voids for media and gas exchange. FIGS. 21A-21D provide schematic examples (not to scale).

FIG. 21A shows a top view of one embodiment of 3DFS. FIG. 21B is a side view of the same scaffold. Stacked nanofiber arrays are visible with defined spacing between the layers. This spacing can be controlled during formation of the 3DFS according to need. For example, the areas containing voids (areas with low-density or no fibers) can be placed to allow media and/or gas exchange to cells proliferating on the higher density areas. The fiber diameter, angle, and spacing can be controlled to facilitate specific culture needs. FIG. 21C is a perspective view of the 3DFS. In this particular embodiment, the 3DFS is cubic, having 15 cm sides a, b, and c. The scaffolds can be made in various sizes according to need, from as small as 0.125 cm³ to larger than 5000 cm³. In some embodiments, the scaffold is not a cube. FIG. 21D provides an example of a 3DFS scaffold engineered to function as a spheroid proliferation cage. In this particular embodiment, the scaffold has a central area for controlled cell proliferation, or spheroid cage. This is achieved by stacking arrays having closely spaced fibers longitudinal fibers at the middle and few or no fibers at the edges. Each layer is stacked such that the fibers are arranged perpendicular to the preceding layer, forming a central cage of closely spaced fibers having an area of about 200 μm in length and width. The outer corners are voids to allow for media and gas exchange.

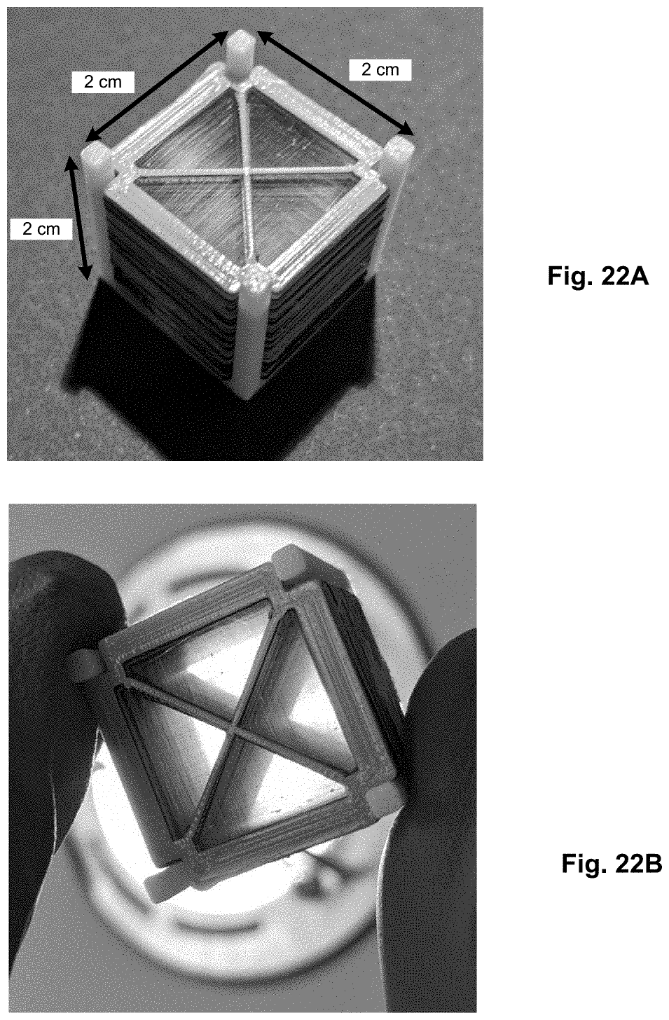

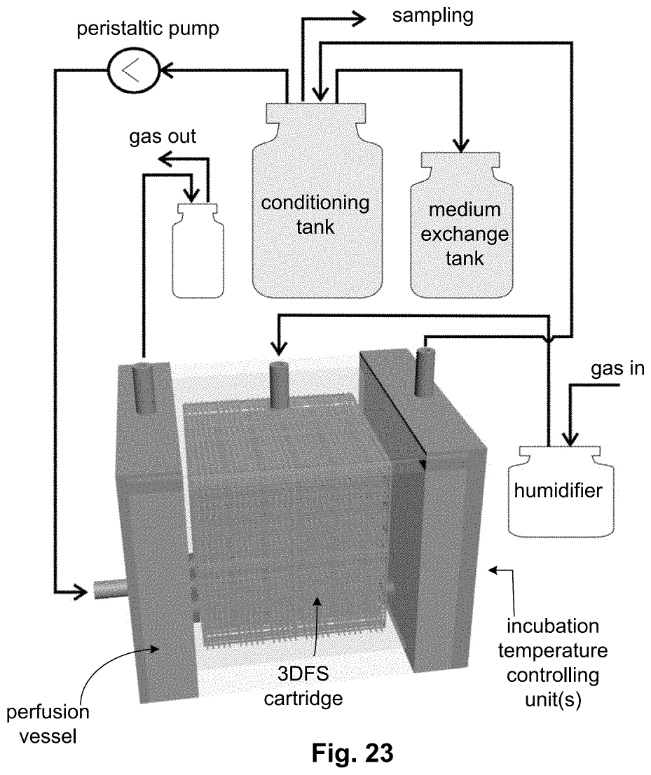

The scaffolds are customizable in terms of precision fiber-fiber spacing (A), fiber-fiber alignment angle (θ), individual fiber diameter and elastic modulus, adjustable micro- and macro-porosity, the spacing between stacked 2D fiber arrays, and the arrangement of individual fibers etc. Individual fibers can be made of different materials and functionalized with biochemical cues (e.g. growth factors and/or proteins decorated on the fibers). These scaffolds can be tailored to have different sizes, e.g., from 0.125 cm³ (FIGS. 22A-22B) to larger than 5000 cm³. These scaffolds can be made of domains with alternating nanofiber densities to generate volumes with a high density of nanofibers (e.g., for growth of cell spheroids) and channels with no nanofibers for the transport of media (as shown in FIG. 21D).

The 3D fiber scaffolds as presented herein can be included in a cell expansion bioreactor. Multiple cubes or cylinders (such as a 5000 cm³) could be combined in cell expansion system to scale-up the cell manufacturing for cell therapy applications with cell manufacturing capacity over 10 billion cells/batch.

The 3DFS can be framed into a cartridge that fits onto a simple perfusion system to media and gases (FIG. 22). The 3DFS can be designed to be plug and play in a perfusion vessel. The vessel can be part of the cell expansion bioreactor. The bioreactor can include a conditioning tank for the cell culture and/or media. The conditioning tank can be connected to the perfusion vessel via a pump (e.g. a peristaltic pump). The bioreactor can also include one or more gas inlets and a gas outlet. Other features can include a medium exchange tank, a humidifier, a heater/incubator for temperature control, a media temperature sensor, pH sensors or other sensors to monitor and/or adjust the culture conditions. Advantageously, the perfusion vessel can be reusable and sterilizable. The perfusion vessel can be custom built to accommodate 3DFS of different sizes.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, “about 0” can refer to 0, 0.001, 0.01, or 0.1. In an embodiment, the term “about” can include traditional rounding according to significant figures of the numerical value. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

What is claimed is:

1. A method of making single filament fibers, comprising:
dispensing a droplet of a polymer solution from a nozzle onto a base, where the nozzle is positioned above the base such that the droplet free falls from the nozzle onto the base, causing the polymer solution to be drawn into a fluid tail between the base and the nozzle; and
as the fluid tail is being drawn, a solvent of the polymer solution evaporates, thereby solidifying the fluid tail to form a nanofiber, where the fiber has a fiber diameter of about 50 nm-100 μm and a fiber length from about 1 cm to 2 m.

2. The method of claim 1, further comprising tuning the fiber diameter and the fiber length by adjusting a distance between the nozzle and the base.

3. A method of forming a nanofiber scaffold, comprising:
forming a first single filament nanofiber by gravitational drawing of a polymer solution from a nozzle to a base, where the polymer solution comprises a polymer and a solvent, where evaporation of the solvent during gravitational drawing causes the polymer solution to solidify into a fiber, wherein each nanofiber independently has a diameter of about 50 nm-100 μm;
collecting the first single filament nanofiber on a collection frame; and
forming and collecting one or more subsequent single filament nanofibers on a collection frame to form a first 2D array layer;
wherein the collecting includes aligning and spacing each single filament nanofiber in the first 2D array layer, wherein an alignment angle between any two adjacent single filament nanofibers is independently selected from about 0 to 179 degrees, and wherein a spacing between any two adjacent fibers is independently selected from about 10 micron or larger.

4. The method of claim 3, further comprising introducing at least one layer of spacer fibers on the 2D array layer, wherein the spacer fiber layer comprises two or more spacer fibers, and wherein the spacer fibers are microfibers or nanofibers.

5. The method of claim 4, further comprising depositing a second 2D array layer on the spacer fiber layer to form a 3D scaffold.

6. The method of claim 5, further comprising alternately forming and collecting additional spacer fiber layers and 2D array layers onto the 3D scaffold.

7. The method of claim 4, wherein each spacer fiber layer or 2D array layer can be rotated in the X-Y plane orthogonal to a fiber orientation in the first 2D array layer.

8. The method of claim 5, wherein the 3D scaffold has a porosity of about 20% to about 99%.

9. The method of claim 3, wherein individual nanofibers in the scaffold have the same or different diameters from one another.

10. The method of claim 3, wherein individual nanofibers have the same or different compositions.

11. The method of claim 4, wherein the spacer fibers have the same or different composition from individual nanofibers.

12. The method of claim 5, wherein the 3D scaffold has a porosity of about 90% to 99%.

13. A device for forming a 3D nanofiber scaffold, comprising:
a fiber collection and delivery assembly;
a fiber drawing assembly comprising a nozzle positioned above a tension stage; and
a fiber positioning and scaffolding assembly comprising a moving stage and a fiber collector frame; and
a cutting tool.

14. The device of claim 13, wherein the fiber collection and delivery assembly comprises fiber positioning arms.

15. The device of claim 13, wherein the fiber collector frame is configured to be rotated about an X-axis.

16. The device of claim 13, wherein the moving stage moves along an X-axis to collect nanofibers to form a 2D array on the fiber collector frame.

17. The device of claim 13, wherein the moving stage moves along a Z-axis to form a 3D scaffold from layers of 2D arrays.

18. The device of claim 13, wherein the fiber scaffolding assembly comprises a supporting frame and at least one stepper motor, wherein the stepper motor drives the fiber to be rotated around the supporting frame.

19. The device of claim 18, wherein the supporting frame is selected from a prism, a cylinder, a pyramid, or an irregular shape.

20. The device of claim 18, wherein the supporting frame is comprised of a soluble material.

21. The device of claim 18, wherein the fiber collection and delivery assembly comprises a storage and delivery wheel.

22. A 3D cell culture scaffold, comprising:
at least two arrays of nanofibers, wherein the each of the nanofibers in the arrays has a diameter between 50 nm and 2 μm;
wherein the nanofibers are formed by gravitational drawing of a polymer solution from a nozzle to a base, where the polymer solution comprises a biocompatible polymer and a solvent, where evaporation of the solvent during gravitational drawing causes the polymer solution to solidify into a fiber;
wherein the nanofibers in the arrays have controlled alignment and spacing;
wherein the arrays of nanofibers are layered to form the scaffold; and
wherein the scaffold has a porosity of 50% or higher.

23. The 3D cell culture scaffold of claim 22, wherein the arrays comprise high-density fiber sections and low-density fiber sections, such that the layered arrays form cell growth areas and media transport areas in the scaffold.

24. The 3D cell culture scaffold of claim 22, wherein the scaffold has a volume of about 0.125 cm³ to 5000 cm³.
